(19) United States
(12) Patent Application Publication
(10) Pub. No.: US 2010/0221707 A1
(43) Pub. Date: Sep. 2, 2010

(54) DIAGNOSTIC METHODS FOR DETECTING CONGENITAL BONE DEFECTS

(75) Inventors: Kenneth E. White, Westfield, IN (US); Lynda F. Bonewald, Kansas City, MO (US); Jian Q. Feng, Dallas, TX (US)

Correspondence Address: BARNES & THORNBURG LLP, 11 SOUTH MERIDIAN, INDIANAPOLIS, IN, 46204, US

(73) Assignee: The Curators of the University of Missouri

(21) Appl. No.: 12/374,700

(22) PCT Filed: Aug. 17, 2007
(86) PCT No.: PCT/US07/76247

§ 371 Date: Apr. 22, 2010

Related U.S. Application Data
(60) Provisional application No. 60/838,257, filed on Aug. 17, 2006.

(51) Int. Cl. C12Q 1/68; C07K 16/00

(52) U.S. Cl. 435/6; 530/387.9

(57) ABSTRACT

The present disclosure is directed to compositions and methods for screening for patients at risk for autosomal recessive hypophosphatemic rickets. More particularly, diagnostic reagents and procedures are provided for analyzing samples to detect defective DMP1 expression.

### CROSS REFERENCE TO RELATED APPLICATIONS

This application claims priority under 35 USC §119(e) to U.S. Provisional Application Ser. No. 60/838,257, filed Aug. 17, 2006, the disclosure of which is incorporated herein by reference.

### BACKGROUND

Mutations in PHEX (see Jonsson, K. B. et al. Fibroblast growth factor 23 in oncogenic osteomalacia and X-linked hypophosphatemia. N Engl J Med 348, 165663 (2003) have been associated with human disorders of phosphate (Pi) handling and skeletal mineralization (causing X-linked hypophosphatemic rickets [XLH]). Such defects are also observed in the Phex-mutant Hyp mouse which results in increased osteocyte expression of the phosphaturic factor FGF23 (see Liu, S. et al. Pathogenic role of Fgf23 in Hyp mice. Am J Physiol Endocrinol Metab 291, E38-49 (2006). Mutations in FGF23 that prevent its degradation also cause autosomal dominant hypophosphatemic rickets (ADHR). Two unrelated, consanguineous kindreds have recently been identified in which affected individuals originally present renal phosphate-wasting, rachitic changes and lower limb deformity, but do not exhibit mutations in any of the previous genes associated with such conditions.

Dentin matrix protein 1 (DMP1) is a highly phosphorylated protein which plays a key role in mineralization of the extracellular matrix and in phosphate homeostasis. More particularly, DMP1 is highly expressed in osteocytes, and when deleted in mice, results in a hypomineralized bone phenotypes (see Ling, Y. et al. DMP1 depletion decreases bone mineralization in vivo: an FTIR imaging analysis. J Bone Miner Res 20, 2169-77 (2005). To date, the full-length DMP1 has not been isolated from bone of various species. Rather, two proteolytically processed fragments of 37 and 57 kDa have been isolated and characterized. The purified, highly phosphorylated 57 kDa C-terminal fragment has been shown to be a hydroxyapatite nucleator in a cell-free system.

Applicants have found that a lack of a properly functioning DMP1 results in defective osteocyte maturation and increased FGF23 expression, leading to pathological changes in bone mineralization. Thus applicants have discovered that a defective DMP1 gene gives rise to the condition known as Autosomal Recessive Hypophosphatemic Rickets (ARHR). More particularly, a lack of functional DMP1 within bone matrix results in defective osteocyte maturation, leading to pathological changes in phosphate homeostasis and in mineralization. Left untreated rickets is often associated with growth retardation, bowing of the lower extremities, and poor dental development. Various methods can be used to treat ARHR once identified in a patient, including the administration of Vitamin D (in its active form, i.e., 1,25 dihydroxycholecalciferol or “Calcitriol”), or by phosphate supplementation. Early detection of patients at risk of developing rickets is desirable to allow early treatment to minimize the impact of the disease.

Several genetic tests are currently available for screening for Hypophosphatemic rickets (autosomal dominant form associated with mutations within the FGF23 gene), Hypophosphatemic rickets (an X-linked form of rickets associated with mutations in the PHEX gene) and Pseudo-vitamin D deficiency rickets (autosomal recessive associated with mutations in the CYP27B1). As disclosed herein applicants' discovery provides an additional gene that should be screened to detect ARHR individuals who would previously not be identified by the existing commercially available test.

### SUMMARY

The present disclosure is directed to diagnostic reagents and procedures for the detection of congenital bone defects. More particularly, the present disclosure is directed to methods for screening patients for autosomal recessive hypophosphatemic rickets (ARHR) resulting from defective Dentin Matrix Protein1 (DMP1) expression. In one embodiment the methods of the present disclosure are used to genetically screen patients for the present of defects in the (DMP1) to diagnose the existence of, or assess the risk of producing offspring that suffer from ARHR.

In accordance with one embodiment, a method of detecting individuals that express a defective DMP1 protein is provided. Such individuals may exhibit ARHR or they may be carriers of the disease. In one embodiment the method comprises sequencing either a portion of, or the entire length of the DMP1 gene isolated from the individual undergoing analysis to identify DMP1 variants. More particularly, in one embodiment the patient's DMP1 sequences are screened to detect a DMP1 variant that has a deletion of nucleic acid sequences 1484-1490 (deletion of CTATCAC; SEQ ID NO: 35) and the presence of the contiguous sequence CCAACTGTGAAGATC (SEQ ID NO: 36).

In another embodiment a kit is provided for screening biological samples for the presence of defective DMP1 genes. In one embodiment the kit comprises a set of PCR primers for amplifying the DMP1 gene, or alternatively the kit comprises one or more sets of PCR primers for amplifying one or more specific regions of the DMP1 gene. The kit may be further provided with reagents for conducting nucleic acid sequencing. In a further embodiment the kit is provided with one or more reagents for conducting PCR reactions. In another embodiment the kit comprises labeled nucleic acid probes that specifically bind to defective DNP1 gene sequences relative to the native DNP1 sequence. In one embodiment the labeled nucleic acid probe binds to the sequence GTTGATGCAACAAACC (SEQ ID NO: 37) under conditions wherein the probe fails to substantially bind (e.g., above background levels) to the sequence GTTGATGCCTATCACAACAAACC (SEQ ID NO: 38). In on embodiment the probe is a 6-10 nucleotide sequence comprising the sequence TGCAAC (SEQ ID NO: 39) or ATGCAACA (SEQ ID NO: 40).

In another embodiment, a method of detecting aberrant DMP1 expression in a patient's cells, as a diagnostic indicator of ARHR, is provided The method comprises contacting proteins of the patient's tissue with an ligand that specifically binds to the peptide of SEQ ID NO: 42 or SEQ ID NO: 43, detecting specific ligand-DMP1 complexes, wherein the formation of ligand-DMP1 complexes indicates a risk of developing ARHR. In one embodiment the ligand is a monoclonal antibody specific for the variant DMP1 protein of SEQ ID NO: 42 or SEQ ID NO: 43.

### BRIEF DESCRIPTION OF THE DRAWINGS

FIG. 1A-1K. DMP1 mutations, osteomalacia, and a defective osteocyte lacunocanalicular network in ARHR. FIG. 1A, Family 1 had a biallelic deletion of nucleotides 1484-1490 in Dmp1 exon 6 (upper trace ARHR patient; lower trace, control individual, missing nucleotides boxed); FIG. 1B, The 1484-1490del segregates with the disorder as assessed by RFLP, creating a new HpyCH4V site, which creates 257 and 84 by fragments from the 341 by exon 6 PCR product (circles: female; square: male, filled symbol: affected; patients F1-1, F1-2 and F1-3 depicted chronologically left to right); FIG. 1C, The 1484-1490del results in a frame shift that deletes the last 18 residues of DMP1 and adds 33 novel residues, with amino acids encoded by the 3′ UTR; FIG. 1D, Family 2 had a start codon mutation (A1>G, upper trace) that resulted in a methionine to valine change (M1V), not present in control individuals (lower trace); FIG. 1E, M1V results in loss of the 16-residue DMP1 signal sequence, due to translational initiation at an internal Met; FIG. 1F, M1V segregates with the ARHR phenotype in Family 2, and creates novel 52 by and 107 by fragments from a 159 by PCR product; FIG. 1G, Wild type (WT) and the ARHR DMP1 mutant expression in HEK293 cells. WT DMP1 was detectable by Western analyses as a 94 kD protein in the cellular lysates, and 94 and 57 kD polypeptides in the cell media. The 1484-1490del mutant was primarily secreted as the 57 kD form of DMP1, with fainter expression in the cellular lysates, whereas the M1V mutant was retained within the cell as the 94 kD form of DMP1, and had no detectable signal in media. FIG. 1H, Goldner staining indicates abundant osteoid (red color) on bone edges (arrowheads), and surrounding osteocytes (arrows). FIGS. 1I through 1J, Resin-casted SEM images show osteocyte lacunae in a cluster (i), with few dendrites and rough surfaces (j). FIG. 1K Serum FGF23 levels: ARHR patients (Mut) compared to heterozygous individuals (Het) and wild type individuals (WT); Family 1 (filled circles), Family 2 (open circles). The upper limit of normal (54 pg/mL) is shown as the dashed line.

FIG. 2A-2I Dmp1-null mice display rickets, osteomalacia and defects in mineralization. FIG. 2A, Serum Fgf23 levels are shown for Dmp1-null (KO) mice compared to the control littermates (Cont). Data are mean±SE from 2-5 month-old mice; n=6 (KO), n=11 (Het); **P<0.01. FIG. 2B, in situ hybridization of Fgf23 shows increased Fgf23 mRNA expression (red in signal) in 10 day old KO osteocytes only. FIG. 2C, Real time RT-PCR of Dmp1-null long bone demonstrates marked elevation of Fgf23 expression, *P<0.05. FIG. 2D, representative radiographs of skeletons from the Cont and KO mice at 3 mo of age. In the KO skeleton, the flared ends of long bones are indicated by arrows and the rachitic rosary of the ribs by an arrowhead. FIG. 2E, Confocal microscopy images of fluorochrome labeling, counter-stained with DAP1 for visualization of osteocyte nuclei. Dmp1-null osteocytes are buried in diffuse fluorochrome label, suggesting a defect in the process of mineral propagation. FIG. 2F, Images of backscattered EM of tibias from 6-wk-old mice (Note: samples were treated with osmium for preserving the cell morphology). FIGS. 2F through 2G, STEM maps of unstained-osmium-free thin sections (<1 μm) from the same tibias of Cont (left) and KO mice (right). With this technology, the convergent electron beam is scanned over a defined area of the sample to obtain mineral (g, black), calcium (FIG. 2H, green), and phosphorus (FIG. 2I, red/white) distribution within matrix.

FIG. 3A-3H. Defective osteoblast to osteocyte differentiation and maturation in the Dmp1 null animal. FIG. 3A, A whole mount X-gal stain of a skeleton from a 8-day-old Dmp1-lacZ knock-in pup. FIG. 3B, DMP1 immunostain of bone matrix surrounding osteocytes. FIG. 3C, An increase in alkaline phosphatase activity in 10-day-old Dmp1-null bone matrix (KO, right). FIG. 3D, Abnormal expression of the type 1 collagen mRNA in the KO osteocytes. FIG. 3E, Highly-expressed E11 protein in all KO osteocytes. FIG. 3F, Visualization of disorganized osteocyte-canalicular system in Dmp1-null mice with porcien red injection compared to the well-organized control osteocytes (left) using confocal microscopy at 40× at 5651610 nm. FIG. 3G, SEM images of the acid-etched, resin-casted osteocyte-canalicular system. Note the differences between the control (left) and the KO (right) in distribution, size and surface of osteocytes. FIG. 3H, TEM (transmission electron microscopy) sagittal-section maps of osteocyte canaliculi and dendrites (Cont, left; KO, right).

FIG. 4A-4C High Pi diet rescues the rickets but not the osteomalacic feature of the Dmp1 null phenotype. FIG. 4A, Restoration of Pi homeostasis by high Pi diet for 4-weeks (left) leads to rescue of rickets in Dmp1-null mice as revealed by autoradiography (right). FIG. 4B, Confirmation of rickets rescue using safranin-O staining of growth plates. FIG. 4C, High Pi diet has a limited effect on the Dmp1-null osteomalacia phenotype. Goldner stain reveals abundant osteoid (red color) is still present on bone edges (arrowhead), and surrounding osteocytes (arrow). von Kossa staining (low power in frame; black, mineral; red, osteoid) validates results using Goldner stain. *Statistically different (*P<0.05).

FIG. 5A-5B Complexity of the osteocyte lacuno-canlicular system. Polished resin embedded mouse alveolar bone (3 mo old) was acid-etched to remove mineral leaving behind the plastic for visualization using SEM (FIG. 5A). This image depicts not only the complexity of the system but how extensively bone is permeated by this network. The insert (FIG. 5B) shows an enlargement of the area outlined in (FIG. 5A).

FIG. 6A-6C. High Phosphate diet does not completely rescue the osteomalacia in the Dmp1 null mice. Double fluorochrome labeling (Alizarin Red, red in signal; calcein, green) of the long bones (2 mo old) shows: FIG. 6A, sharp discrete mineralization front in control; FIG. 6B, diffuse labeling in Dmp1 null mice with normal diet; FIG. 6C, some rescue, but still osteomalacia in Dmp1 null mice fed a high Pi diet.

FIG. 7 represents a schematic drawing of the DMP1 expression constructs. Full-length DMPI (FL), mutant with a cleavage site mutated at amino acid 213 (D-to-A), 37 kDa N-terminal fragment (aa17 to aa212) (37KN), and long (57KL) and short (57Ks) forms of 57 kDa C-terminal fragment (aa206 to aa503, and aa250 to aa503, respectively) were cloned downstream of the cytomegalovirus (CMV) promoter in the pcDNA3 (Invitrogen) expression vector, separately. There is a 45 amino acid residue difference between the 57KL and the 57Ks. The endogenous DMPI signal peptide aa1 to aa16 (MKTVILLVFLWGLSCAL; SEQ ID NO: 46) was linked to both 57 kDa fragments. The mutant form of DMP1 (D213A) was generated by Dr. Chunlin Qin at University of Texas Houston Health Science Center Dental Branch

FIG. 8 represents data obtained from Stains-All staining and Western-blot analysis of recombinant DMP1 expression. Various pcDNA3-DMPI expression constructs, including pcDNA3 vector (E), full-length DMPI (FL), mutant (M), 37 kDa N-terminal fragment (37^{N}), and long and short forms of 57 kDa C-terminal fragment (57L and 57s) were transiently transfected into the expression cell line 293EBNA cells using Lipofectamine 2000 reagent (Invitrogen). Twenty-four hours after transfection, the medium was replaced by serum-free medium and cultured for an additional 48 hours. Twenty microliters of conditioned medium was loaded on a 4-20% gradient gel, and the proteins were visualized with Stains-All (A) or detected by western-blot using an antibody 784 generated against the N-terminal peptide 116-136 (B), which recognizes both the full-length DMPI as well as the 37 kDa N-terminal fragment, or antibody 785 generated against the C-terminal peptide 485-499 (C), which recognizes both the full-length DMPI as well as the 57 kDa C-terminal fragment.

FIG. 9 represents data showing the effects of overexpression of full-length DMP1. Representative radiographs of tibiae show no apparent phenotype in mice overexpressing Col1a1-DMPI at ages of 1 month (A), 2 months (B) and 5 months (C), compared to the age-matched Dmp1 heterozygous mice (HET).

FIG. 10A-10B represents data showing the effects of re-expression of full-length DMP1 in Dmp1-null mice using the Col1a1 promoter. A. In situ hybridization (upper panel) indicated that the Col1a1-DMPI transgene (red staining) was highly expressed in osteoblasts lining the bone surface in the Dmp 1 heterozygous mice carrying the Col1a1-DMPI transgene (HET/Tg) as well as in the Dmp1-null mice carrying the Col1a1-DMPI transgene (RES, rescued), compared with the Dmp1 heterozygous (HET) control. No Dmp1 mRNA was detected in the Dmp1-null mice (KO, knock-out). B. Immunohistochemical localization of DMPI protein (lower panel) showed that there was much higher DMPI protein (in brown color) present in the matrix surrounding the osteoblasts as well as osteocytes in both HET/Tg and RES, suggesting a long half-life of the DMPI protein. In contrast, DMPI protein was mainly localized in the matrix surrounding the osteocytes in HET mice. No DMPI protein was observed in the KO mice. Scale bar is 20 um.

FIGS. 11A & 11B represents data showing the effects of re-expression of full-length DMP1 in Dmp1-null osteoblasts/osteocytes. A) Radiographs of tibiae show that the skeletal abnormalities are rescued in Dmp1-null mice with targeted re-expression of the full-length DMPI (RES), compared to the Dmp1 heterozygous mice (HET) and Dmp1-null mice (KO), at ages of 1 month, and 2 months and 5 months. B) The quantified data show that the length of tibia in Dmp1-null mice is rescued by targeted-expression of full-length DMPI. N=4. *, p<0.05; **, p<0.01; ***, p<0.001. C) Safranin-O staining shows that the growth plate defects are rescued by targeted expression of full-length DMPI in Dmp1-KO mice at age of 2 months. Scale bar is 1 mm in A, 500 um in C.

FIG. 12 represents fluorochrome labeling in mice with targeted re-expression of full-length DMP1. Fluorochrome-labeled sections of 2-month-old ulnae reveal sharp, distinct labeling lines in the heterozygous control mice (HET, left panel, green arrow head), diffuse labeling in the Dmp I-null mice (KO, central panel, red arrow head), and restoration of double sharp labeling lines in DmpI-null mice with re-expression of the full-length DMPI in cells of the osteoblast lineage (RES, right panel, green arrow head). Scale bar is 10 um.

FIG. 13 represents photographs of the lacuno-canalicular system in mice with targeted re-expression of full-length DMP1. Panel A, Procion red, a small molecular dye which diffuses through the lacunocanalicular system, was injected through the tail vein. Confocal images of the osteocytecanalicular system filled with procion red reveal the lacuno-canalicular systems in the Dmp 1 heterozygous mice (REI, left), Dmp1-null mice (KO, middle), and Dmp1-null mice with targeted re-expression of the full-length DMPI (RES, right). Panel B shows the resin-casted SEM images of the lacuno-canalicunar system in the Dmp1 heterozygous mice (REI, left), Dmp1-null mice (KO, middle), and rescued mice (RES, left). Note that the lacunocanalicular systems in the KO mice are disorganized with few branches, whereas the morphology of the Dmp1-null osteocyte-canalicular system is restored in the rescued mice.

FIG. 14 demonstrates the immunolocalization of the E11 protein in wild type, Dmp1 null mice and Dmp1 null mice having the DMP1 gene re-introduced. Immunohistochemistry showed that E11, a membrane protein, was restricted to the early osteocytes in the heterozygous control mice (HET). However, E11 was expressed in almost all Dmp1-null osteocytes (KG). Reexpression of the full-length DMPI in Dmp1-null mice (RES) restored the pattern of the E11 expression to the early osteocytes, suggesting that the osteoblast differentiation defects were rescued. Scale bar is 0.1 um.

FIG. 15 represents data showing the effects of overexpression of the DMP1-57 kDa C-terminal fragment. A. In situ hybridization shows high levels of the Col1a1-DMP1 transgene expression in the osteoblasts (black arrows) lining the bone surface (Col1a1-57K, right panel, red staining), compared to age matched control mice, where endogenous Dmp1 is mainly expressed in osteocytes (white arrows) (WT, left panel). B. Immunohistochemical localization of DMP1 protein shows high levels of 57 kDa fragment in the matrix surrounding osteoblasts and osteocytes in Col1a157K transgenic mice (right panel, brown color), compared to the age-matched control where endogenous DMP1 is detected in m˜trix surrounding osteocytes only (left panel). Radiographs show no apparent phenotype in the lower limbs from 3-month-old Col1a1-57K transgenic mice, compared to the WT in the same litter (C). Scale bar in A and B is 50 um. Scale bar in C is 1 mm.

FIG. 16 represents data showing the effects of re-expression of the DMP1-57 kDa C-terminal fragment in Dmp1 null mice. The sections of tibiae were prepared from 3-week-old mice. In situ hybridization (A) and immunohistochemistry (B) showed that expression of DMPI in Dmp1 heterozygous mice (HET) was found predominantly in the osteocytes (white arrow) embedded in the bone matrix. No expression of endogenous DMPI was detected in Dmp1-null mice (KO). In situ hybridization showed that the Col1a1-57K transgene was highly expressed in osteoblasts (black arrows), but the immunohistochemistry indicated that the 57 kDa fragment was present in the matrix surrounding the osteoblasts and osteocytes in Dmp1-null mice with targeted re-expression of the 57 kDa fragment (57K-RES). Scale bar is 50 um.

FIG. 17 represents data showing the effects of re-expression of the DMP1-57 kDa C-terminal fragment on the skeletal abnormalities of Dmp1 null mice. Representative radiographs of the tibiae show that re-expression of the 57 kDa fragment rescues the skeletal abnormalities of Dmp I-null mice at ages of 10 days (A), 3 weeks (B), and 7 weeks (C). The quantified data shows that the length of tibia is rescued in Dmp1-null mice with targeted-expression of 57 kDa fragment at age of 7 weeks (D). N=6. ***, p<0.001. Scale bar is 1 mm in A, Band C.

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “isolated” as used herein refers to material that has been removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “purified” and like terms relate to the isolation of a molecule or compound in a form that is substantially free of contaminants normally associated with the molecule or compound in a native or natural environment.

As used herein, the term “antibody” refers to a polyclonal or monoclonal antibody or a binding fragment thereof such as Fab, F(ab′)₂ and Fv fragments.

The term “label” as used herein refers to any atom or molecule which can be used to provide a detectable (preferably quantifiable) “signal”, and which can be attached to a nucleic acid or protein. Labels may provide “signals” detectable by fluorescence, radioactivity, colorimetry, gravimetry, X-ray diffraction or absorption, magnetism, enzymatic activity, and the like.

As used herein, “stringent conditions” refers to hybridization conditions and/or amplification conditions in which a probe or primer will specifically hybridize to a target nucleic acid while not binding substantially to non-target nucleic acids. “Stringent conditions” typically involve hybridizing at about 50oC to about 68oC in 5×SSC/5×Denhardt's solution/1.0% SDS, and washing in 0.2×SSC/0.1% SDS at about 60oC to about 68° C.

As used herein the term “congenital bone defect” is intended to include altered skeletal mineralization and/or disturbed inorganic phosphate homeostasis, optionally associated with increased FGF23 production.

As used herein the term “osteomalacia” refers to abnormal softening of bones caused by deficiencies of phosphorus or calcium or vitamin D.

As used herein the term “defective Dentin Matrix Protein 1 (DMP1)” relates to any DMP1 protein that differs from the wild type amino acid sequence and fails to adequately perform its native biological function, resulting in the onset of ARHR. The defective DMP1 may be the result of a deletion, insertion or inversion in the DMP1 gene sequence, or a nucleic acid modification that result in frameshift or nonsense mutations in the encoded mRNA or impact the regulatory sequences of the gene. A defective DMP1 gene is a gene that encodes a defective DMP1.

As used herein the term “defective Dentin Matrix Protein1 (DMP1) expression” is intended to encompass the complete loss of DMP1 expression, or a reduction in the level of expression of DMP1 protein, the expression of a defective DMP1 or the improper localization of DMP1, including the failure to deliver the protein to its native location.

### EMBODIMENTS

Applicants have discovered that the function of a single protein, Dentin Matrix Protein 1 (DMP1), is required to allow proper formation of bone. More particularly, a lack of DMP1 within bone matrix results in defective osteocyte maturation in patients, leading to pathological changes in phosphate homeostasis and in mineralization. Accordingly, patients with a defective DMP1 protein develop Autosomal Recessive Hypophosphatemic Rickets (ARHR), which if left untreated is associated with growth retardation, bowing of the lower extremities, and poor dental development. Various methods can be used to treat ARHR once the condition has been identified, including the administration of Vitamin D (in its active form, i.e., 1,25 dihydroxycholecalciferol or “Calcitriol”), and/or phosphate supplementation. Early detection of patients at risk of developing ARHR is desirable to allow early treatment to minimize the impact of the disease.

One aspect of the present disclosure is directed to compositions and methods for screening for the presence of defective DMP1 protein production in patients. Although ARHR is a recessive trait, identification of individuals that harbor one defective MP1 gene can be beneficial to advise individuals of their risk of producing a child that will have ARHR. The detection of defective DMP1 genes and proteins can be conducted using any of the standard analytical techniques known to those skilled in the art for detecting nucleic acid and amino acid variants. More particularly, for detecting variant DMP1 gene sequences, nucleic acid sequencing, nucleic acid hybridization probes, restriction fragment polymophisms, PCR amplification and melting curve analysis and other techniques known to those skilled in the art can be used. For detecting variant DMP1 proteins, the use of amino acid sequencing techniques, including but not limited to mass spectrometry analysis, enzymatic activity or monoclonal antibodies specific for DMP1 variants and other techniques known to those skilled in the art can be used.

In accordance with one embodiment the method of detecting defective DMP1 expression comprises isolating a biological sample from a patient, recovering genomic DNA from the biological sample and analyzing the genomic DNA for the presence of variant DMP1 sequences. The patient can be a human or other vertebrate species. The biological sample can be any tissue or bodily fluid that comprises intact cells, and in one embodiment the biological sample comprise blood or epidermal cells, including cells recovered by buccal swabs. In one embodiment the method comprises amplifying nucleic acids recovered from the patient using suitable PCR primers. Typically the nucleic acid is DNA and more specifically genomic DNA. The primers can be selected such that only a smaller region of interest is amplified relative to the entire DMP1 gene sequence. In one embodiment the PCR primers are selected immediately upstream and downstream of the sequence of SEQ ID NO: 39 or SEQ ID NO: 44. In accordance with one embodiment, melting curve analysis is conducted during the PCR amplification procedure to identify the presence of heterozygous or homozygous mutated DMP1 genes.

In an alternative embodiment a PCR reaction can be conducted in situ on thin slices of a biological tissue sample recovered from a patient. In situ reactions offer the advantage of demonstrating the location and the amount of DMP1 nucleic acid sequences in the various cell compartments, and may provide prognostic information as well as help define treatment strategies. In situ analysis can also be conducted using labeled monoclonal antibodies that are specific for proteins expressed by defective DMP1 genes.

Alternatively, mutated DMP1 nucleic acid sequences are identified by determining the sequence of either a portion of, or the entire coding sequence of, the DMP1 gene. In one embodiment, when the analysis involves only sequencing a portion of the DMP1 gene, the region selected either includes the nucleotidesequences surrounding the start codon or the region responsible for encoding the 57 kDa peptide fragment of DMP1, including the region from +1474 to +1500. In another embodiment the promoter region of the DMP1 gene is sequenced. The sequencing of the DMP1 gene or DMP1 gene fragments can be conducted using standard techniques with or without PCR amplification.

In accordance with one embodiment, the present disclosure is directed to diagnostic reagents and procedures for the detection of congenital bone defects. More particularly, the present disclosure is directed to methods for screening patients for autosomal recessive hypophosphatemic rickets (ARHR) resulting from defective Dentin Matrix Protein1 (DMP1) expression. In one embodiment the methods of the present disclosure are used to genetically screen patients for the present of defects in the (DMP1) to diagnose the existence of, or assess the risk of producing offspring that suffer from ARHR.

In accordance with one embodiment, a method of detecting individuals that express a defective DMP1 protein is provided wherein a nucleic acid probe is used to identify defective DMP1 genes. In one embodiment the nucleic acid probe is selected such that the probe hybridizes to defective DMP1 sequences but not to wild type DMP1 sequences, particularly under stringent conditions. The nucleic acid probe can be labeled to detect binding, or the probe may comprise a member of a pair of PCR primers such that successful amplification of the nucleic acid segment indicates the presence of the variant DMP1 sequence. In one embodiment a patient's nucleic acid sequences are screened for the presence of DMP1 genes that have a deletion of nucleic acid sequences 1484-1490 (i.e., deletion of CTATCAC; SEQ ID NO: 35) and accordingly, the presence of the contiguous sequence CCAACTGTGAAGATC (SEQ ID NO: 36). In accordance with one embodiment the probe comprises the sequence of SEQ ID NO: 39 or SEQ ID No: 40.

In another embodiment a kit is provided for screening biological samples for the presence of defective DMP1 genes. In one embodiment the kit comprises a set of PCR primers for amplifying the DMP1 gene, or alternatively the kit comprises one or more sets of PCR primers for amplifying one or more specific regions of the DMP1 gene. The kit may be further provided with reagents for conducting nucleic acid sequencing. In a further embodiment the kit is provided with one or more reagents for conducting PCR reactions. The kit can be further provided with instructional materials, additional reagents and disposable labware for conducting PCR amplifications or nucleic acid sequencing reactions.

The reagents of the kit may include buffers and/or a DNA polymerase enzyme. In one embodiment the kit is provided with thermostable polymerase such as the Taq polymerase, for example. In another embodiment the PCR primers provided with the kit are labeled, or reagents are provided for labeling the PCR primer or detecting the amplification product of the reaction. The nucleic acids and other reagents can be packaged in a variety of containers, e.g., vials, tubes, bottles, and the like. Other reagents can be included in separate containers and provided with the kit; e.g., positive control samples, negative control samples, buffers, etc.

In another embodiment the kit comprises labeled nucleic acid probes that specifically bind to defective DNP1 gene sequences relative to the native DNP1 sequence. In one embodiment the labeled nucleic acid probe binds to the sequence GTTGATGCAACAAACC (SEQ ID NO: 37) under conditions wherein the probe fails to substantially bind (e.g., above background levels) to the sequence GTTGATGCCTATCACAACAAACC (SEQ ID NO: 38). In on embodiment the probe is a 6-10 nucleotide sequence comprising the sequence TGCAAC (SEQ ID NO: 39) or ATGCAACA (SEQ ID NO: 40).

In another embodiment, a method of detecting aberrant DMP1 expression in a patient's cells, as a diagnostic indicator of ARHR, is provided The method comprises contacting proteins of the patient's tissue with an ligand that specifically binds to the peptide of SEQ ID NO: 42 or SEQ ID NO: 43, detecting specific ligand-DMP1 complexes, wherein the formation of ligand-DMP1 complexes indicates a risk of developing ARHR. In one embodiment the ligand is a monoclonal antibody that specifically binds the variant DMP1 protein of SEQ ID NO: 42 or SEQ ID NO: 43.

In accordance with one embodiment an antibody is provided that specifically binds to a defective DMP1 protein. In a further embodiment the antibody is a monoclonal antibody. In one embodiment a monoclonal antibody is provided that specifically binds to the peptide of SEQ ID NO: 42, more particularly, a monoclonal antibody that binds to the peptide sequence of SEQ ID NO: 45.

It is contemplated that any antibody or probe used in the present disclosure will be labeled with a “reporter molecule,” which provides a detectable signal. The label may include, but is not limited to fluorescent, enzymatic (e.g., ELISA, as well as enzyme-based histochemical assays), radioactive, and luminescent systems. It is not intended that the present invention be limited to any particular detection system or label.

### Example 1

### Loss of DMP1/DMP1 Causes Rickets and Osteomalacia: Role of the Osteocyte in Mineral Metabolism

The potential for Dentin Matrix Protein 1 (DMP1) to direct skeletal mineralization and to regulate phosphate (Pi) homeostasis was investigated. DMP1 is highly expressed in osteocytes, and when deleted in mice, results in a hypomineralized bone phenotypes.

### Methods

ARHR patients: All patients were provided written, informed consent in accord with the Indiana University and the Children's Hospital of Eastern Ontario Institutional Review Boards. Both kindreds were of Lebanese descent. Serum FGF23 was assessed with an Intact FGF23 ELISA (Kainos, Inc.; Tokyo, Japan). Bone biopsies were assessed for osteomalacia using Goldner's stain and fluorescence microscopy using standard protocols (see Ling, Y. et al., J Bone Miner Res 20, 2169-77 (2005) and Glorieux, F. H. et al., Bone 26, 103-9 (2000)).

Genomic DNA extracted from blood samples was PCR-amplified and assessed by DNA sequencing for each DMP1 exon. The cDNAs of the WT human DMP1 and both mutants were subcloned into pcDNA3.1(+)V5/His vector (Invitrogen) to create the V5 and 6×His-tagged expression constructs and transiently transfected into HEK293 cells for Western blot analyses. Protein samples, and standards for molecular mass determination, were electrophoresed on 15% SDS-PAGE mini-gels (Bio-Rad Inc. Hercules, Calif.) and electrotransferred onto PVDF membranes (Bio-Rad Inc., Hercules, Calif.). Membranes were incubated with 0.25 μg/ml of an HRP-conjugated anti-V5 antibody (Invitrogen, Inc.). Blots were visualized by enhanced chemiluminescence (ECL) (Amersham Inc., Piscataway, N.J.). Control transfections consisting of vector alone demonstrated no reacting bands.

Serum and urine concentrations of calcium, phosphorus, creatinine and serum alkaline phosphatase activity were measured using standard methods. Serum intact PTH was determined by immunoradiometric assay (N-tact*; Incstar Corp., Stillwater, Minn., USA). 1,25-(OH)₂D levels were measured using radioimmunoassays (1,25-Dihydroxyvitamin D Osteo SP; Incstar Corp., Stillwater, Minn., USA). All samples were obtained fasting. TmP/GFR was calculated according to the Walton and Bijvoet nomogram (Feng J Q, et al., The Dentin Matrix Protein 1 (Dmp1) is Specifically Expressed in Mineralized, but not Soft Tissues during Development. Journal of Dental Research (2003)) using a 2 hour urine sample and a serum sample that was obtained after 1 hour.

Mice: All animal studies were in accord with the guidelines of the University of Missouri-Kansas City animal review board. The Dmp1-null mice were generated with exon 6 deleted as described previously (Feng, J. Q. et al., Bone Miner Res 17, 1822-31 (2002). A CD-1 background was used in this study. Previous examination of a mixed background of C57BL/6 and 129 Sv found the same skeletal phenotype, regardless of strain. Furthermore, there were no apparent differences between the Het and WT mice in any parameters measured to date.

Diet: The mice were fed autoclaved Purina rodent chow (5010, Ralston Purina Co., St. Louis, Mo.) containing 1% calcium, 0.67% phosphorus, and 4.4 IU vitamin D/g (regular diet). To normalize the blood Pi level of the Dmp1-null mice, the animals were fed a rescue chow (Harlan TEKLAD, Cat. TD.87133) containing 2% phosphorus, 1.1% calcium, 2.2 IU/g vitamin D from 21 days of age.

Preparation and analyses of bone samples: Procedures for bone sample preparation and high resolution X-ray, TEM, and SEM were described previously McKee, et al., Anat Rec 234, 479-92 (1992). For STEM or TEM images the thin sections were cut and stained with uranyl acetate and lead citrate and examined using a Philips CM12 in STEM mode. For resin-carted osteocyte-lacuno-canalicular SEM, the surface of methylmethacrylate embedded bone was polished followed by acid etching with 37% phosphoric acid for 2-10 seconds, 5% sodium hypochlorite for 5 minutes, then coated with gold and palladium, and examined by FEI/Philips XL30 Field emission environmental SEM (Hillsboro, Oreg.). Standard methods for safranin-O staining growth plates, Goldner's Masson Trichrome staining, immunohistochemistry, and in situ hybridization using digoxigenin-labeled cRNA probes have been described previously (Feng, J. Q. et al., J Bone Miner Res 17, 1822-31 (2002). To analyze the role of osteocytes in mineralization, mice were injected 5 days apart with calcein (5 mg/kg i.p), alizarin red (20 mg/kg i.p) and again with calcein. The animals were sacrificed 2 days after the final injection. The 50 mm non-decalcified samples from these animals were photographed using a Nikon PCM-2000 confocal microscope coupled to an Eclipse E-800 upright microscope for fluorochrome labeling combined with DAPI staining of nuclei of osteocytes. Bone activity of alkaline phosphatase was performed according manufacturer's instructions (Sigma).

Visualization of the osteocyte-canalicular system by procion red: The small molecular weight dye (0.8%, 0.01 ml/g, Sigma) was injected through the mouse tail vein while under anesthesia using Avertin (5 mg/kg body weight) 10 min before sacrifice. The fresh bone was fixed in 70% ETOH and sectioned to 50 μm for photography using confocal microscopy.

Quantification of mRNA: Measurement of FGF-23 mRNA was performed using fluorescent labeled TaqMan MGB primers (Forward: CTG CTA GAG CCT ATC CGG AC (SEQ ID NO: 1); Reverse AGT GAT GCT TCT GCG ACA A (SEQ ID NO: 2)), combined with iTaq CYBR with a ROX detection Kit (Bio-Rad, Hercules, Calif.). Real-time detection of GAPDH mRNA signal (Forward: GGT GTG AAC CAC GAG AAA TA (SEQ ID NO: 3); Reverse: TGA AGT CGC AGG AGA CAA CC (SEQ ID NO: 4) was also performed as the internal control for the amplification of FGF-23 mRNA. Data were collected quantitatively and the CT number was corrected by CT readings of corresponding GAPDH controls. Data were then expressed as fold changes compared to experimental controls.

Serum and urine assays: Serum and urine calcium were measured using a colorimetric calcium kit (Stanbio Laboratory, Boerne, Tex.). Serum and urine phosphorus were measured by the phosphomolybdate-ascorbic acid method as previously described. Serum FGF23, 1,25-(OH)₂D, and PTH levels were measured by a full-length FGF-23 ELISA kit (Kainos Laboratories, Inc. Tokyo, Japan), a 1,25-Dihydroxy vitamin D EIA kit (Immunodiagnostic Systems Limited, Boldon, UK), and a mouse intact PTH ELISA kit (Immutopics, Carlsbad, Calif.), respectively. Urine samples were collected in mouse metabolic cages for 16 hours and a renal phosphorus clearance (RPC) was calculated with the (urine Pi X urinary volume)/(serum Pi X time of collection) as previously described (Rowe, P. S. et al., Genomics 67, 54-68 (2000). Urine creatinine was measured with a Creatinine Assay Kit (Cayman Chemical Company, Ann Arbor, Mich.).

### Results

Two unrelated, consanguineous kindreds in which affected individuals originally presented with renal phosphate-wasting, rachitic changes and lower limb deformity were investigated. In Family 1 (F1), there were three affected sisters (F1-1, F1-2 and F1-3), and in Family 2 (F2), there was a single affected female (F2-1). The parents and siblings of these individuals showed no clinical or biochemical evidence of the condition.

Patients F1-1 and F1-3 presented with rickets and progressive lower limb deformity in late infancy, whereas sister F1-2 was noted to have rachitic changes on a chest x-ray at age 7 months. In contrast, F2-1 presented with a mild genu valgum at 8 years of age. The pre- or off treatment age-related metabolic profiles for both kindreds were similar, characterized by hypophosphatemia due to renal phosphate-wasting (serum Pi 0.7-0.9 mmol/L, normal: 1.2-1.8; TmP/GFR 0.61-0.81 mmol/L, lower limit of normal ≧1.0), high normal to moderately elevated alkaline phosphatase, normal intact parathyroid hormone (PTH) levels (4.6-6.9 pmol/L, normal: 1.6-6.9), normocalcemia (ionized calcium 1.16-1.18 mmol/L, normal: 1.1-1.3) and eucalciuria (urinary calcium to creatinine ratio 0.19-0.33, normal ≦0.6) (see Table 1).

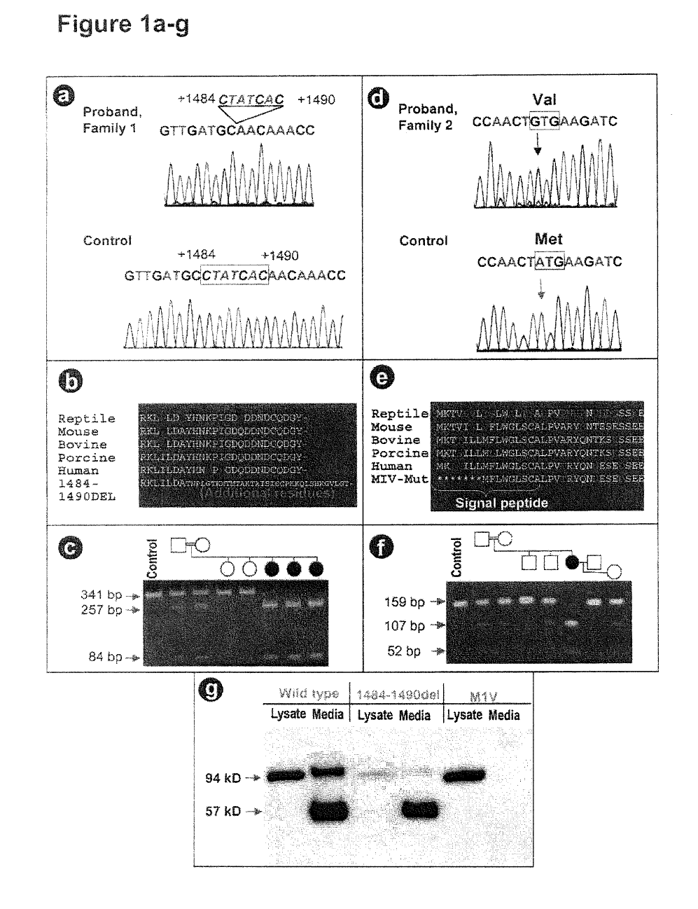

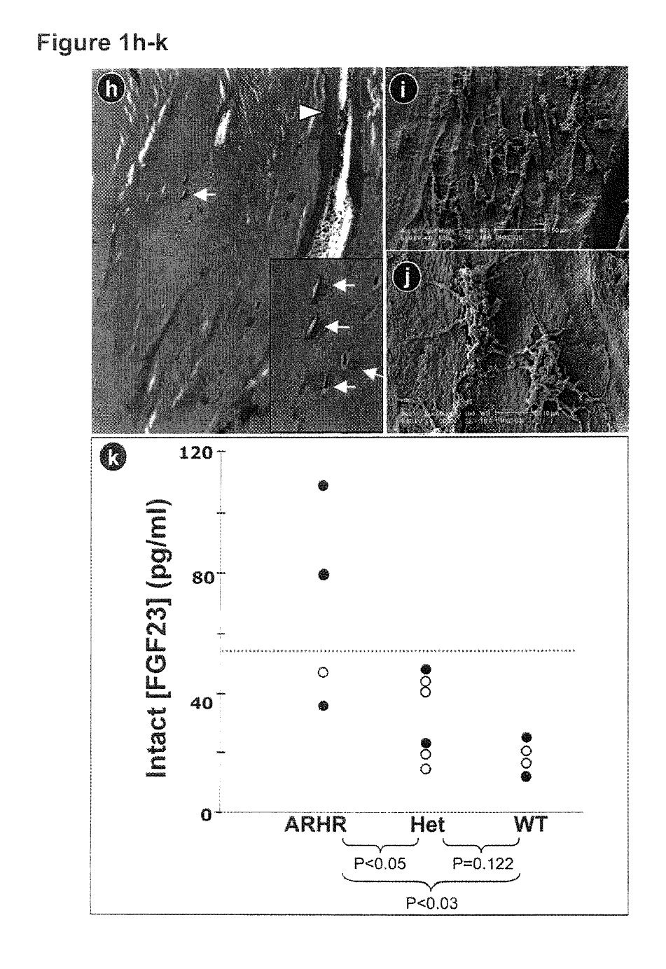

TABLE 1
Comparisons of Biochemistry Data for ARHR and Dmp1-null Mouse

| Human ARHR | | | | | Mouse Dmp1-null | | | |
|---|---|---|---|---|---|---|---|---|
| | | | | SAMPLE | VALUE | | | |
| | VALUE | | AGE | SIZE | Mean ± S.E. | AGE | | SAMPLE SIZE |
| Ionized Calcium | F1-1 | 1.21 (N: 1.1-1.3) | 21 years | 3 | 2.30 ± 0.05 (2.45 ± 0.05)* | 2 | weeks | 11 (12) |
| (ARHR) mmol/L | F1-2 | NA | NA | | 2.00 ± 0.05 (2.13 ± 0.05)* | 7 | weeks | 5 (7) |
| | F1-3 | 1.16 | 5 years | | 1.90 ± 0.05 (1.85 ± 0.05) | 4-6 | months | 7 (11) |
| | F2-1 | 1.19 | 32 years | | | | | |
| Total Calcium | | | | | | | | |
| (Dmp1-null mice) | | | | | | | | |
| mmol/L | | | | | | | | |
| Phosphorus | F1-1 | 0.9 (N: 1.2-1.8) | 7 years | 4 | 1.94 ± 0.06 (2.97 ± 0.10)** | 2 | weeks | 11 (12) |
| mmol/L | F1-2 | 0.7 | 11 months | | 1.18 ± 0.14 (2.3 5 ± 0.09)** | 7 | weeks | 5 (7) |
| | F1-3 | 0.8 | 5 years | | 1.26 ± 0.13 (1.94 ± 0.06)** | 4-6 | months | 11 (14) |
| | F2-1 | 0.9 | 9 years | | | | | |
| TmP/GFR | F1-1 | 0.81 (N: ≧1.0) | 7 years | 4 | 0.052 ± 0.007 (0.032 ± 0.003)* | 4-6 | months | 6 (6) |
| (ARHR) mmol/L | F1-2 | 0.62 | 11 months | | | | | |
| | F1-3 | 0.61 | 9 years | | | | | |
| | F2-1 | 0.78 | 9 years | | | | | |
| Renal Pi Clearance | | | | | | | | |
| (Dmp1-null mice) | | | | | | | | |
| mL/min | | | | | | | | |
| Intact PTH | F1-1 | 6.9 (N: 1.6-6.9) | 21 years | 3 | 56.5 ± 9.5 (4.9 ± 1.9)** | 8 | weeks | 7 (10) |
| pmol/L | F1-2 | NA | NA | | 23.0 ± 2.6 (4.4 ± 0.5)** | 4-6 | months | 6 (9) |
| | F1-3 | 4.9 | 5 years | | | | | |
| | F2-1 | 4.6 | 32 years | | | | | |
| 1, 25 (OH)₂D | F1-1 | 74 (N: 40-140) | 21 years | 3 | 82.2 ± 25.9 (114 ± 25.4) | 8 | weeks | 4 (5) |
| pmol/L | F1-2 | NA | NA | | | | | |
| | F1-3 | 71 | 5 years | | | | | |
| | F2-1 | 77 | 32 years | | | | | |
| Alkaline Phosphatase | F1-1 | 276 (N: 150-380) | 7 years | 4 | 267.42 ± 81.03 (100.46 ± 43.70) | 8 | weeks | 4 (6) |
| U/L | F1-2 | 836 | 11 months | | | | | |
| | F1-3 | 269 | 5 years | | | | | |
| | F2-1 | 330 | 9 years | | | | | |
| FGF23 | See FIG. 1c | | | | 8381 ± 807 (122 ± 16.5)** | 2 | weeks | 7 (7) |
| pg/ml | | | | | 713 ± 121 (43 ± 5)** | 7 | weeks | 5 (7) |
| | | | | | 864 ± 96.6 (100 ± 8.0)** | 4-6 | months | 6 (11) |
| Urinary Calcium/ | 1-1 | 0.20 (N: ≦0.6) | 7 years | 4 | 0.13 ± 0.02 (0.12 ± 0.02) | 4-6 | months | 5 (5) |
| Creatinine | F1-2 | 0.33 | 11 months | | | | | |
| mol/molCr | F1-3 | 0.19 | 9 years | | | | | |
| | F2-1 | 0.06 | 9 years | | | | | |

N = normal ranges for age
NA = not available
For the Dmp1-null mouse data, controls are in ( )
*= p < 0.05
**= p < 0.01

Serum 1,25(OH)₂D levels, available in 3 patients, were inappropriately normal for the degree of hypophosphatemia when measured at >4 years (71-77 pmol/L, normal: 40-140). Resolution of rickets and normalization of alkaline phosphatase were observed during treatment with phosphate supplementation and calcitriol; however, the TmP/GFR remained low. Linear growth trajectories were heterogeneous among the affected individuals: patients in F1 had a mid-parental height of 154.5 cm (5th to 10th percentiles) with F1-1 and F1-2 measuring 153 (5th percentile) and 136.5 (<5th percentile) cm at final adult height, respectively. FI-3 had a height of 153.5 cm at 10 months post-menarche, well within the genetic target. The patient in F-2 had a final adult height of 172 cm (90-95th percentile), 3 cm above the upper limit of her genetic target. Both families were negative for FGF23 and PHEX mutations. These two kindreds were thus preliminarily designated as having autosomal recessive hypophosphatemic rickets (ARHR), and were distinguished from HHRH by the presence of eucalciuria.

The ARHR families were tested for mutations in DMP1, a protein that is primarily expressed in mineralized tissues. A homozygous deletion of nucleotides 1484-1490 (1484-1490del) in DMP1 exon 6 (FIG. 1a) was detected in the first kindred, which resulted in a frame shift that replaced the conserved C-terminal 18 residues with 33 novel residues (FIG. 1b). The second kindred had a biallelic nucleotide substitution within the DMP1 start codon (ATG to GTG, or A1>G) (FIG. 1d), which resulted in substitution of the initial methionine with valine (M1V), present in exon 2 (FIG. 1e). These mutations segregated with the disorder in both kindreds (FIGS. 1c,f), and neither DMP1 mutation was found in 206 control alleles. Following transfection into HEK293 cells and Western blotting using an anti-V5 antibody, normal DMP1 carrying a C-terminal V5-tag was detectable within the cellular lysates, as well as secreted into the cell media (FIG. 1g). In contrast, the 1484-1490del mutant was faint, yet detectable in the cellular lysates and was highly elevated in the media. The M1V mutant, however, was wholly retained within the cells, consistent with loss of the signal peptide due to translational initiation at an internal methionine (FIG. 1g).

A trans-ilial biopsy in patient F1-3 confirmed severe osteomalacia (osteoid thickness 17.1 μm, normal: 5.9±1.1 SD; mineralization lag time 56.3 days, normal: 14.1±4.3 SD), and increased bone volume per tissue volume (43.2 percent, normal 22.4±4.2 SD). Excessive osteoid was observed not only within cutting cones (arrow head) but also surrounding osteocyte lacunae (arrows) (FIG. 1h). The peri-lacunar hypomineralized regions were mainly present on the side of the lacunae oriented towards the central canal of the osteon. The osteocyte lacuno-canalicular system was characterized by rough surfaces with few canaliculi (FIG. 1i-j). Elevation of serum FGF23 was observed in the ARHR patients, all receiving calcitriol and phosphate therapy (FIG. 1k). Similar to X-linked hypophosphatemic rickets (XLH), the ARHR patient serum FGF23 values overlap with the upper normal range.

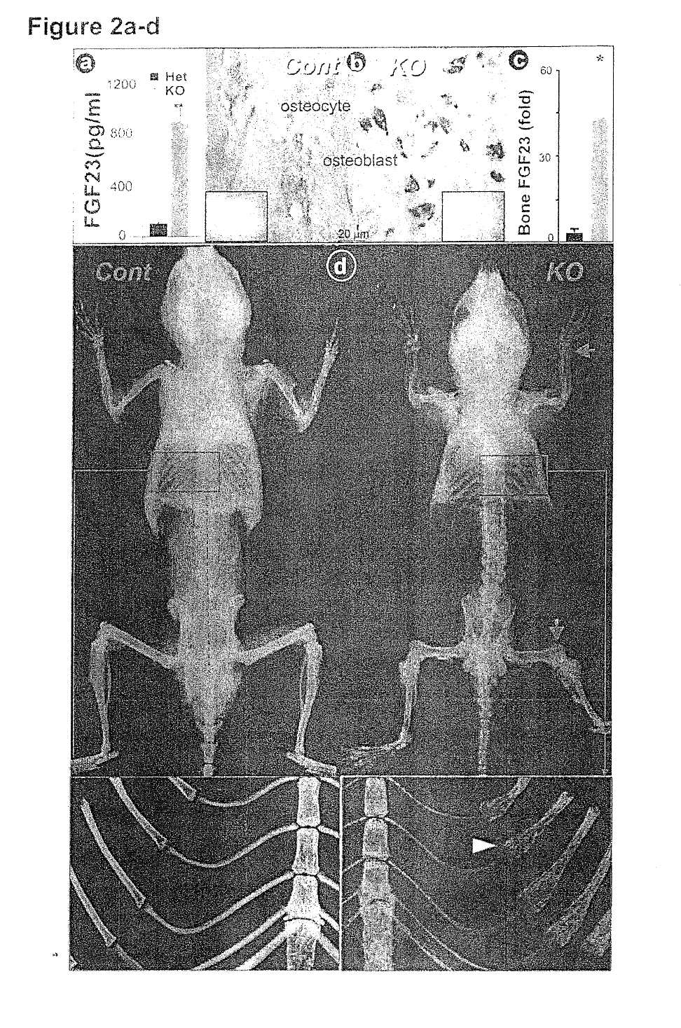

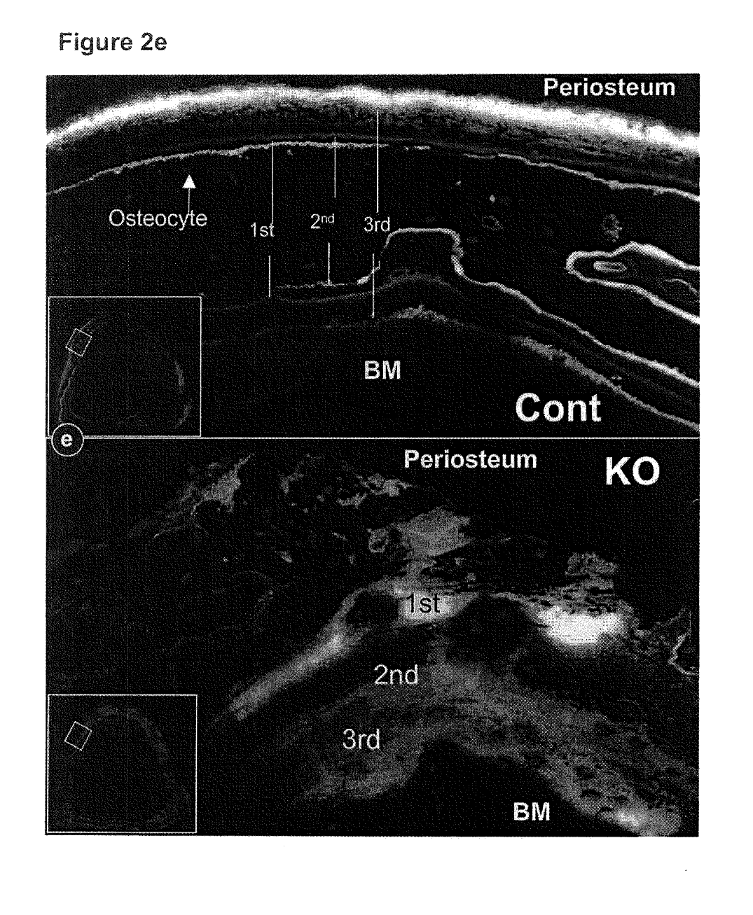

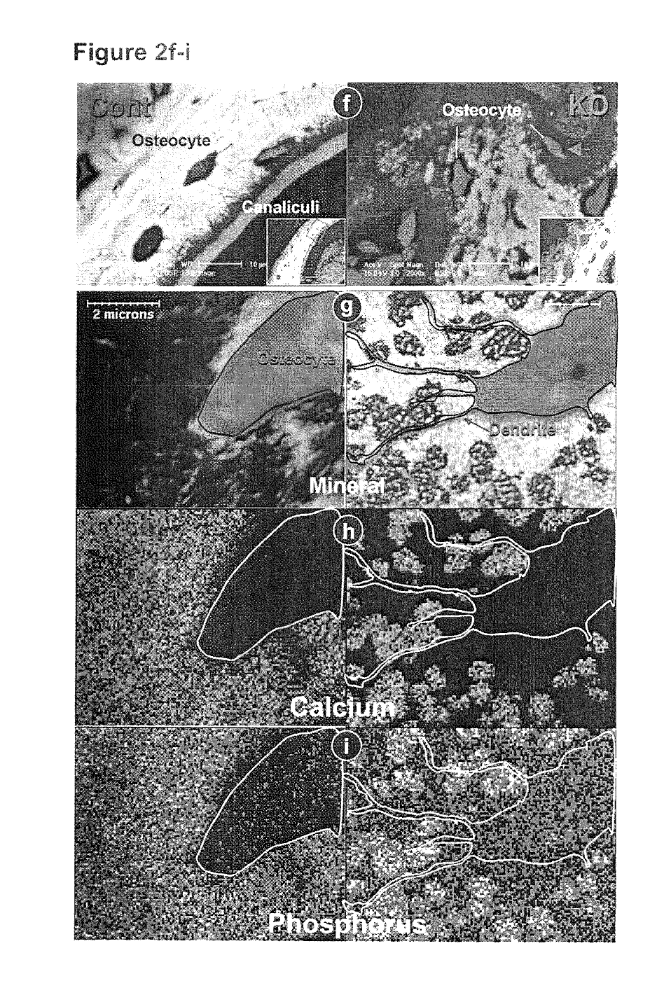

Toyosawa and colleagues had previously shown that DMP1 was highly expressed in the osteocyte (Toyosawa, S. et al. Dentin matrix protein 1 is predominantly expressed in chicken and rat osteocytes but not in osteoblasts. J Bone Miner Res 16, 2017-26. (2001). To determine if lack of Dmp1 in osteocytes could be responsible for both the human and murine phenotype, and to test the potential mechanism of action, the observed abnormalities of mineralization in this mouse were investigated to determine whether they were associated with renal Pi wasting and increased FGF23 production by osteocytes. As reported previously, the Dmp1-null mice are mildly hypocalcemic, but severely hypophosphatemic (control mice 6.0±0.2 mg/dL;. Dmp1-null (null) 3.9±0.4; Ling, Y. et al. DMP1 depletion decreases bone mineralization in vivo: an FTIR imaging analysis. J Bone Miner Res 20, 2169-77 (2005). Studies showed increased renal phosphorus clearance (RPC) (control 0.032±0.003 mL/min; null 0.052±0.007) as well as increased PTH (control 41±5.0 pg/ml; null 216±24.1), and elevated serum FGF23 levels (FIG. 2a). No significant differences were observed in 1,25(OH)₂D levels (control 114±25.4; null 82.2±25.9), although the values in the Dmp1 null serum were lower.

This biochemical profile is similar to that observed in the Hyp mouse model of XLH. The elevated circulating FGF23 concentration in Dmp1-null mice was associated with increased Fgf23 message levels in osteocytes by in situ hybridization (FIG. 2b) and increased bone Fgf23 message expression by real-time PCR (FIG. 2c). DMP1 has been demonstrated as playing an important role in normal dentinogenesis chondrogenesis, and mineralization in vivo (see Ye, L. et al. Deletion of dentin matrix protein-1 leads to a partial failure of maturation of predentin into dentin, hypomineralization, and expanded cavities of pulp and root canal during postnatal tooth development. J Biol Chem 279,19141-8 (2004); Ye, L. et al. Dmp1-deficient Mice Display Severe Defects in Cartilage Formation Responsible for a Chondrodysplasia-like Phenotype. J Biol Chem 280, 6197-203 (2005) and Ling, Y. et al. DMP1 depletion decreases bone mineralization in vivo: an FTIR imaging analysis. J Bone Miner Res 20, 2169-77 (2005), respectively). Newborn mice lacking Dmp1 have no apparent phenotype; but develop the radiological appearance of rickets (FIG. 2d) and an osteomalacia phenotype with age.

Fluorochrome labeling was combined with DAPI nuclear staining for visualization of the position of osteocytes relative to mineralization fronts (FIG. 2e). The control bone showed three discrete lines of fluorescent label, reflecting the mineralization fronts at the time of injection, and a typical pattern of osteocyte nuclei between fronts (FIG. 2e, upper). In contrast, in the Dmp1-null mice, the fluorescent labeling of exposed sites of hydroxyapatite occurred in numerous dispersed, punctate areas surrounding the osteocyte nuclei, reminiscent of a diffuse, osteomalacic form of mineralization (FIG. 2e, lower). Using backscattered scanning electronic microscopy (SEM), mineral was evenly distributed surrounding the osteocyte lacunae in the control bone (FIG. 2f, left, white), however, the mineral content was either missing, or sparsely located in regions surrounding Dmp1-null osteocytes (FIG. 2f, right, arrows). Scanning transmission electron microscopy (STEM) was then employed to obtain a more accurate localization of mineral in relation to osteocytes. In control mice, the mineralized matrix (black, 2g), calcium (green, 2h), and phosphorus (red, 2i) surrounding the osteocyte were evenly distributed in the surrounding bone matrix (FIG. 2g-i, left). In contrast, in Dmp1-null mice, spherical structures reminiscent of calculospherulites were present with markedly reduced propagation into the surrounding osteoid (FIG. 2g-i, right).

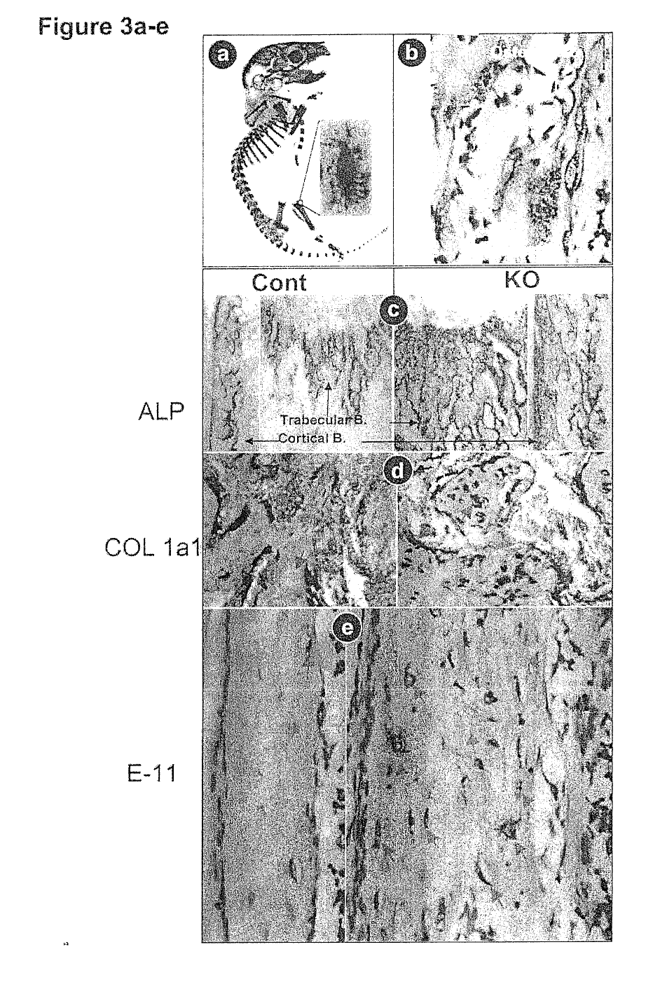

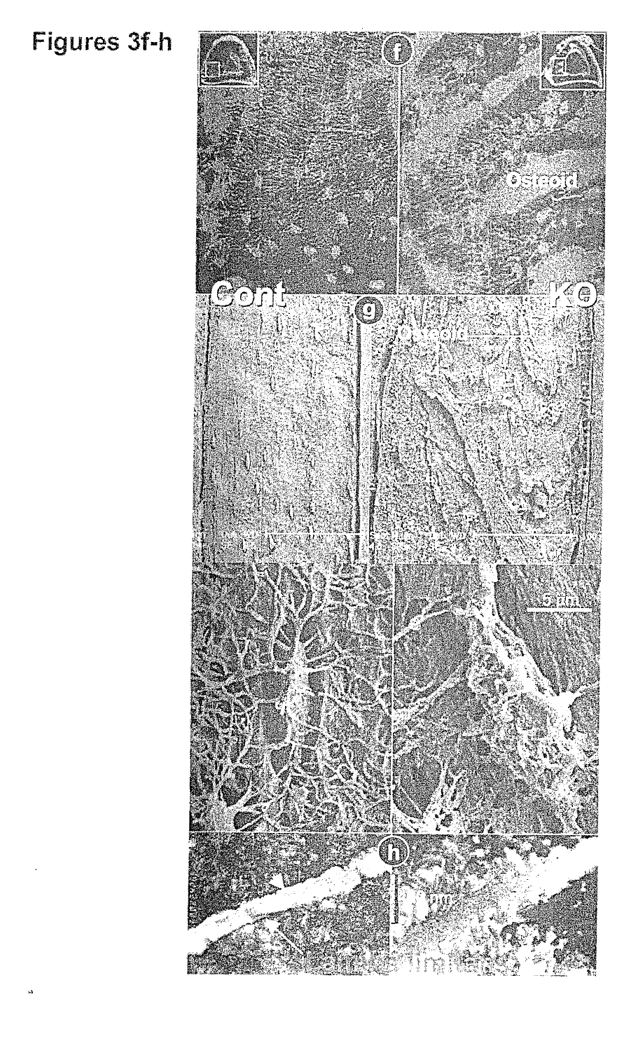

High expression of Dmp1-lacZ was also observed in osteocytes using 8 day old Dmp1-lacZ knock-out mice (FIG. 3a, left). Immunohistochemical staining of 4 month old control mice showed high expression of DMP1 along osteocyte dendrites/canaliculi, but undetectable expression in osteoblasts (FIG. 3b, right). Studies were undertaken to determine if defective osteoblast maturation and differentiation into osteocytes was responsible for the observed skeletal phenotype of Dmp1-null mice. In contrast to the control, markers of osteoblasts, such as alkaline phosphatase activity (FIG. 3c) and collagen type 1 mRNA (FIG. 3d), as well as early osteocyte markers such as E11/gp38 protein (FIG. 3e) were elevated in the Dmp1-null osteocytes regardless of whether they were newly-formed or deeply embedded. These critical observations partially explain the abnormal skeletal phenotype of Dmp1-null mice as being a result of continued, and inappropriate, expression of osteoblast and osteoid-osteocyte proteins in embedded osteocytes. This suggests that DMP1 expression in the extracellular matrix is essential for normal osteoblast to osteocyte differentiation through down-regulation of osteoblast markers.

The distinguishing morphological feature of osteocytes is their long dendritic processes that travel through canaliculi, where DMP1 is restricted along canalicular walls and within the lamina limitans. To examine the effects of Dmp1 ablation on the lacuno-canalicular system, procian red, a small molecular weight dye which allows tracing of the entire osteocyte lacuno-canalicular system, was delivered via tail vein injection. Confocal microscopy after injection revealed that the control osteocyte lacunae were highly organized and regularly spaced in linear arrays (FIG. 3f, left), whereas the Dmp1-null osteocyte lacunae were much larger, and randomly oriented (FIG. 3e, right). Striking abnormalities in the distribution and organization of the Dmp1-null osteocyte-lacuno-canalicular system were further documented with acid-etched SEM images (FIG. 3g). Indeed, the inner lacuno-canalicular wall was smooth in control sections (FIG. 3g, left); however, the wall was buckled and enlarged in the null mouse (FIG. 3g, right), similar to observations in the patient samples (FIG. 1i-j), and consistent with TEM of the collapsed matrix surrounding the cell and its processes (FIG. 3h, right). The control TEM image (FIG. 3h, left) showed a distinct lamina limitans demarcating the canalicular wall (arrow) and a normal dendritic process, with a distinct, visible space between the dendrite membrane and the canalicular wall. In the poorly mineralized matrix from the Dmp1-null mice (right), unmineralized collagen fibrils were evident with an absence of the lamina limitans, and the membrane surface was buckled and irregular. Taken together, the above data indicate that the osteoid-osteocyte (and perhaps the mature osteocyte) plays an important role in matrix mineralization, and that DMP1 is integral to these functions.

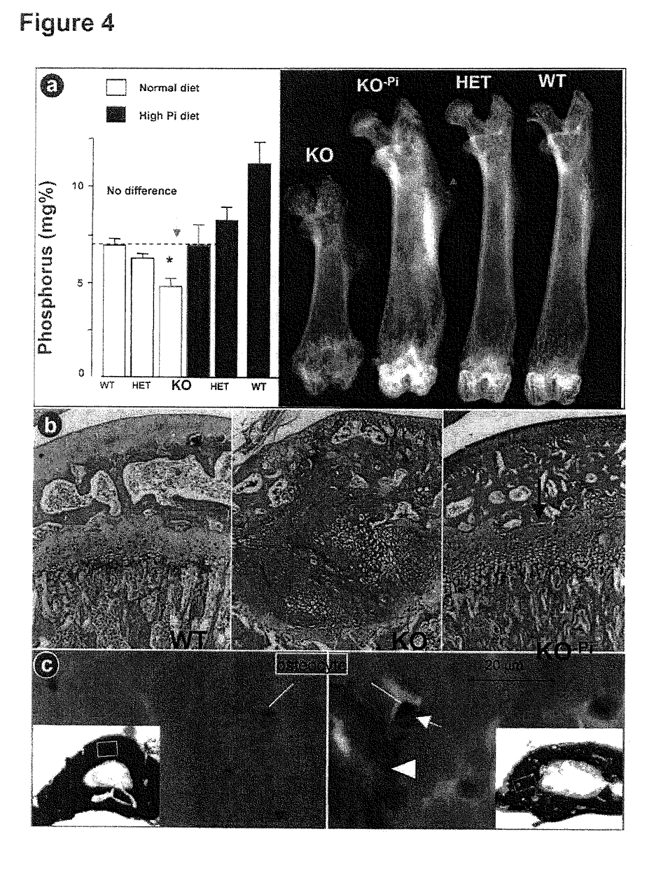

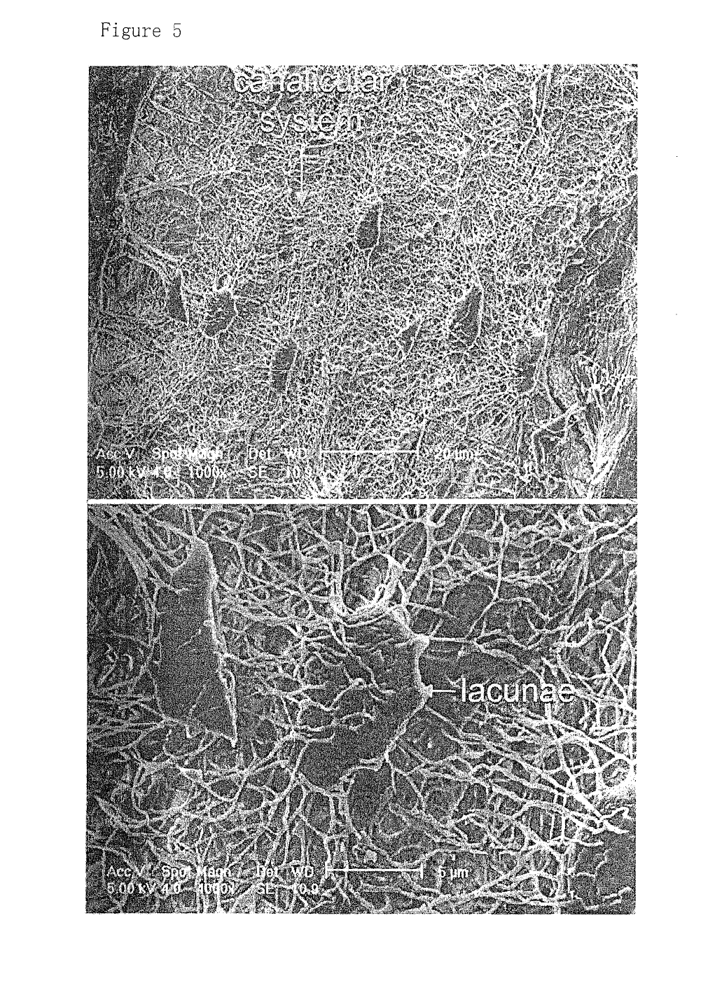

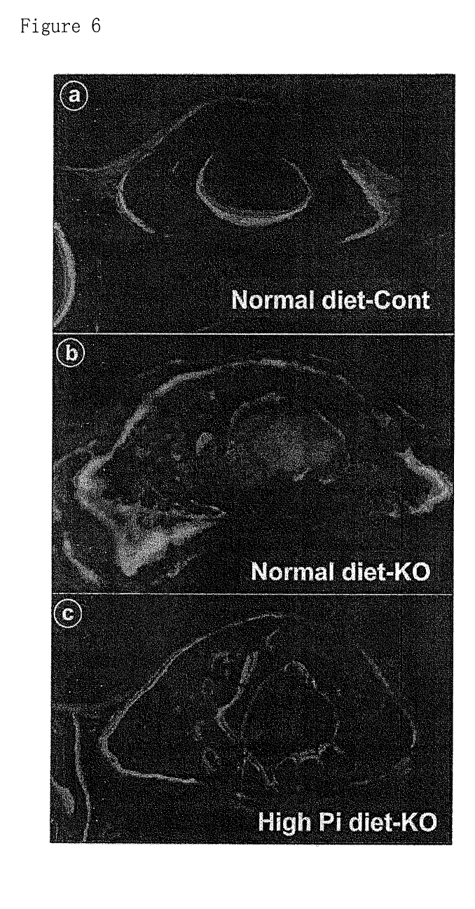

Hypophosphatemia is one of the most prominent defects in Dmp1-null mice. To determine the effects of restoring the serum Pi to control levels, a high Pi diet (2%, Harlan Teklad) was fed to these mice (FIG. 4a, left). This diet rescued the radiological appearance of rickets (FIG. 4a, right), due to a correction of the mineralization defect at the level of the Dmp1-null growth plate (FIG. 4b, right) with marked improvement in the bone formation rate (FIG. 6). Although the osteomalacia improved with Pi diet, the bone phenotype was not completely rescued (FIG. 4c, right and FIG. 6). These observations are consistent with similar Pi supplementation rescue studies in Hyp mice and in human vitamin D-resistant rickets, where complete healing of the rachitic phenotype occurs with only partial resolution of the osteomalacia. Taken together, these data suggest that the rickets feature of this phenotype is due to the hypophosphatemia, whereas the majority of the osteomalacia is due a lack of functional DMP1 in the osteocyte and its microenvironment resulting in defective mineralization. Therefore, these observations suggest both direct (effect on osteocytes) and indirect effects (through phosphate) of DMP1 on mineralization.

In summary, these studies define novel functional roles for the osteocyte, and demonstrate that DMP1 plays an important role in osteocyte maturation. These observations are highlighted by the findings that genetic removal of Dmp1 from the skeletal matrix (in mice) and loss-of-function DMP1 mutations (in human ARHR kindreds) concurrently lead to independently-altered skeletal mineralization and disturbed Pi homeostasis associated with increased FGF23 production, both due to defective osteocyte function.

### Example 2

### The Biological Activity of DMP1 is Primarily Located in the 57 KDA Carboxy-Terminal Fragment: Rescue of the DMP1-Null Phenotype

Dentin matrix protein 1 (DMP1) is a highly phosphorylated protein which plays a key role in mineralization of the extracellular matrix and in phosphate homeostasis. To date, the full-length DMP1 has not been isolated from bone of various species. Rather, two proteolytically processed fragments of 37 and 57 kDa have been isolated and characterized. The purified, highly phosphorylated 57 kDa C-terminal fragment has been shown to be a hydroxyapatite nucleator in a cell-free system. Different forms of DMP1 were transiently expressed in 293EBNA (non-osteocyte) cells. Western-blot analysis showed that full-length DMP1 is processed into 37 kDa and 57 kDa fragments in vitro. Transgenic mice overexpressing the full-length DMP1 or 57 kDa fragment in the osteoblast lineage were then generated using a type I collagen (Col1a1) promoter. Re-expression of the full-length DMP1 in Dmp1-null mice rescues the skeletal abnormalities as determined using multiple approaches, such as radiography, fluorochrome labeling, scanning electron microscopy (SEM) and immunohistochemistry. More importantly, targeted expression of the 57 kDa fragment in cells of the osteoblast lineage also rescued the skeletal abnormalities, but with some time delay depending on the mouse line generated. The phenotype was corrected between 1 week and 1 month of age. Thus, applicants conclude that the 57 kDa fragment recapitulates most, if not all, features of the full-length DMP1.

### Materials and Methods

Generation of Col1a1-DMP1 Transgene, Col1a1-57K Transgene and Various DMP1 Expression Constructs: The coding region for murine DMP1 was obtained by PCR amplification, using the following primers, forward primer (109U): (109˜130) 5′-GAATCGCATCCCAATATGAAGA-3′ (SEQ ID NO: 5); and reverse primer (1659L): (1659˜4680) 5′-CCCAAGACTCCGTCCTGTGAGA-3′ (SEQ ID NO: 6; the gene bank accession number is U65020). The length of the PCR product was 1572 base pairs. The PCR product was subsequently cloned into the TA cloning vector pCR2.1 (Invitrogen, Carlsbad, Calif., USA). The DMP1 cDNA was then released from pCR2.1 vector by XhoI and XbaI endonucleases, and subcloned into XhoI and SpeI sites of the pGL3-Basic vector (Promega, Madison, Wis., USA) to replace the luciferase gene and ligate the DMP1 cDNA with the following SV40 late poly(A) signal. The DMP1 cDNA and SV40 Poly(A) signal fragment was subsequently subcloned into the EcoRV and SalI sites of the mammalian expression vector pBC KS+ (a kind gift from Dr. Barbara E. Kream, University of Connecticut Health Center, Farmington, Conn., USA), containing the 3.6-kb rat type I collagen promoter plus a 1.6-kb intron I, giving rise to the Col1a1-DMP1 transgene.

There are two major cleavage sites in DMP1, which give rise to two 57 kDa fragments with 45 amino acid difference. To generate these two DMP1 57 kDa C-terminal fragment constructs, two rounds of PCR were performed. The gene segment encoding DMP1 signal peptide (16 amino acid residues) was incorporated into the forward primers. Therefore the final PCR product contained the DNA sequence encoding the DMP1 signal peptide in frame with the N-terminus of each gene fragment. In the first round PCR reaction, murine DMP1 cDNA was used as a template, and the following 3 primers were used, the forward primer 1,5′-TTGGGGGCTGTCCTGTGCTCTCGATGATGAAGGGATGCAGAG-3′ (SEQ ID NO: 7) for the long 57 kDa C-terminal fragment (57K^{L}), the forward primer 2,5′-TTGGGGGCTGTCCTGTGCTCTCGATGACAGCCAGTCTGTG-3′ (SEQ ID NO: 8 for shorter 57 kDa C-terminal fragment (57K^{S}) and the same reverse primer 1, corresponding to the end of SV40 late poly A signal (5′-CATCGGTCGACGGATCCTTATC-3′; SEQ ID NO: 9).

In the second round PCR reaction, two 57 kDa fragments generated in the first round PCR were used as a template separately with the following two primers, the forward primer 3, containing the DMP1 signal peptide (5′-GAAT CGATATCCCAATATGAAGACTGTCATTCTCCTIGTGTTCCTTTGGGGGCTG TCCTGTGC-3′; SEQ ID NO: 10) and the same reverse primer 1 used in the first round PCR. The forward primer 3 contained an EcoRV endonuclease cleavage site and the reverse primer contains a SalI endonuclease cleavage site. The final PCR products were digested with EcoRV and SalI restriction enzymes and cloned into the EcoRV/SalI sites of the pBC3.6/1.6 GFP expression vector in place of the GFP gene, giving rise to the Col1a1-57K transgene.

The coding regions of the full-length DMP1 as well as the two 57 kDa C-terminal fragments were also subcloned into the pcDNA3 expression vector, downstream of the CMV promoter, respectively. The DMP1 37 kDa N-terminal fragment was amplified by using the following two primers, forward primer 4,5′-GTGATATCGAATCGCATCCCAATATGAAGA-3′; SEQ ID NO: 11, and reverse primer 2,5′-TCTCTAGAGCTAGCTCTGCATCCCTTCATCAT-3′; SEQ ID NO: 12. The PCR products were then digested with restriction enzymes EcoRV and XbaI, and cloned into the EcoRV/XbaI sites of the pcDNA3 expression vector, giving rise to the pcDNA3-37K construct. A DMP1 construct with the cleavage site mutated at D213A was kindly provided by Dr. Chunlin Qin at University of Texas Houston Health Science Center Dental Branch, Houston, Tex., USA. The mutant DMP1 was subcloned into the EcoRI site of the pcDNA3 expression vector, giving rise to the pcDNA3-mDMP1 construct.

All PCR reactions were performed using high fidelity TaKara Ex Taq™ DNA Polymerase (Takara, Madison, Wis., USA) and the PCR products were confirmed by DNA sequencing (ABI Model 377) at the biotechnology support facility at KU Medical Center, Kansas City, Kans., USA. Both the Col1a1-DMP1 transgene and the Col1a1-57K transgene were released by SacII and SalI from the vector backbone and purified using a Qiaquick® gel extraction kit according to manufacturer's instructions (Qiagen, Valencia, Calif., USA).

### Cell Culture and Transfection

293EBNA cells, a human kidney cell line, and CHO cells, derived from Chinese hamster ovary, were obtained from the American Type Culture Collection (ATCC). Both types of cells were cultured in Dulbecco's modification of Eagles's medium (DMEM, with 4.5 g/L D-glucose, L-glutamine, and sodium pyruvate) (Mediatech, Inc., Herndon, Va., USA) supplemented with 10% Fetal Bovine Serum (FBS, Invitrogen, Carlsbad, Calif., USA). The osteoblast cell line 2T3, derived from transgenic mice expressing SV40 large T antigen under the control of the osteocalcin promoter (a gift from Dr. Stephen E. Harris, University of Texas Health Science Center at San Antonio, San Antonio, Tex., USA), was maintained in minimal essential medium, alpha modification (α-MEM), supplemented with 10% FBS, and 100 IU/ml penicillin, 100 μg/ml streptomycin. Transfection was done using lipofectamine 2000 reagents (Invitrogen, Carlsbad, Calif., USA) according to the manufacturer's instructions. All cell cultures were incubated at 37° C. in a humidified 5% CO₂ atmosphere.

### Stains-All Staining:

Stains-All staining was used to analyze the various forms of the recombinant DMP1 expressed in 293EBNA cells, as described previously (Qin et al. 2003b). Briefly, proteins from serum-free conditioned media were electrophoresed using 4-20% gradient polyacrylamide gels (Life gels, Frenchs Forest NSW, Australia). The gel was then fixed in 45% methanol: 10% acetic acid: 45% H₂O for 1 hour and washed in 50% methanol for 4 hours. The gel was stained overnight in freshly prepared Stains-All solution, containing 0.01% Stains-All (Sigma-Aldrich, St. Louis, Mo., USA), 5% (v/v) formamide, 25% (v/v) isopropanol and 15 mM Tris•CL (pH 8.8). On the next day, the gel was washed with 40% methanol and photographed using a flat bed scanner (HP Scanjet 5490C scanner).

### Western Blotting:

To determine whether the various forms of the recombinant DMP1 were secreted from the cells, the pcDNA3 expression constructs, encoding either full-length wild-type, mutant DMP1, a 37 kDa N-terminal fragment or a 57 kDa long or short form of the C-terminal fragment, were transiently transfected into 293EBNA cells. The growth medium was replaced with serum-free medium 24 hours after transfection and the transfected cells were further cultured for an additional 48 hours. The conditioned medium was collected and centrifuged at 14,000×g for 15 minutes to remove cells and cellular debris. Western blotting was performed as described previously (Gorski, Liu, Artigues, Castagna and Osdoby 2002), using affinity-purified rabbit anti-mouse DMP1 peptide polyclonal antibody. Antibody 784 was raised against the N-terminal peptide 116-136 (GLGPEEGQWGGPSKLDSDEDS; SEQ ID NO: 13), antibody 785 against the C-terminal peptide 485-499 (AYHNKPIGDQDDNDC; SEQ ID NO: 14). Briefly, the conditioned media derived from 293EBNA cells or CHO cells were directly electrophoresed using 4-20% gradient polyacrylamide gels (Life gels, Frenchs Forest NSW, Australia). The conditioned media derived from 2T3 cells were concentrated 10 folds using Millipore Centricon YM-3 concentrator, with a molecular weight cutoff of 3,000 NMWL (normal molecular weight limit) (Millipore, Billerica, Mass., USA) before electrophoresis. The separated proteins were transblotted onto Polyvinylidene fluoride (PVDF) membranes. The membrane was blocked using 5% BioRad blocking grade milk in 1×Tris-buffered saline Tween-20 (TBST), containing 10 mM Tris•HCL, 150 mM NaCL and 0.05% Tween 20 (pH7.5). The blot was incubated overnight with affinity-purified rabbit anti-mouse DMP1 primary antibody (1:4000 dilution) in 1×TBST containing 5% BioRad blocking grade milk, washed and then incubated with horseradish peroxidase (HRP)-conjugated goat anti-rabbit secondary antibody (BioRad, Hercules, Calif., USA) diluted to 1/100,000 in 15 ml of 5% BioRad milk in 1×TBST for a minimum of 1 hour. Immunostained bands were visualized using ECL™ Chemiluminescent Western Blotting Detection Reagents (Amersham Biosciences, Pittsburgh, Pa., USA), according to manufacturer's instructions. Chemiluminescent bands were imaged using a CL-XPosure Film (Pierce Biotechnology, Inc., Rockford, Ill., USA).

Generation of Col1a1-DMP1 and Col1a1-57K Transgenic Mice:

Transgenic mice were generated at the University of Texas-Houston. Briefly, the purified Col1a1-DMP1 transgene or the Col1a1-57K transgene was microinjected into fertilized C57B/L6 eggs at a DNA concentration of 3 ng/μl. Surviving eggs were transferred into the oviducts of pseudopregnant C57B/L6-recipient mice to obtain transgenic mice expressing the DMP1 transgene. The transgenic mice were screened by PCR analysis using DNA extracted from tail biopsy. The transgenic lines were maintained on a C57B/L6 background. Two out of four independent Col1a1-DMP1 transgenic mouse lines and 3 out of ten independent Col1a1-57K transgenic mouse lines were partially characterized and crossed to Dmp1-null mice for rescue studies (see below). The animal use protocol was reviewed and approved by the Institutional Animal Care and Use Committee of the University of Missouri at Kansas City.

Expression of the Col1a1-DMP1 Transgene or Col1a1-57K-L Transgene in Dmp1-Null Mice:

The generation of mice null for Dmp1 using the lacZ knock-in targeting approach has been previously described (Feng et al. 2003). For re-expression of full-length DMP1 in mice lacking Dmp1, female Col1a1-DMP1 transgenic mice were first crossed with homozygous male Dmp1-null mice (viable and fertile) to generate female mice heterozygous for both Col1a1-DMP1 transgene and Dmp1 gene, Col1a1-DMP1^{+/-}; Dmp1^{+/-}. The double heterozygous mice were further bred with Dmp1-null males to produce Dmp1-null mutants carrying the transgene, Dmp1^{-/-}; Col1a1-DMP1^{+/-}. (It is of note that female Dmp1-null mice are fertile but produce small litter sizes.) As no phenotypic differences between the wild-type mice and heterozygous Dmp1-null mice were found (Ye et al. 2004), the heterozygous Dmp1-null mice were used for the control. Five developmental stages were analyzed. Samples were obtained from newborn, 10-day-old, 1-month-old, 2-month-old, and 5-month-old mice for this study. All mice were bred to C57B/L6 background. The same breeding strategy was used to introduce the Col1a1-57K-L transgene into Dmp1-null mice.

### Tail PCR Genotyping:

The genomic DNA was extracted from tail biopsy and used for genotyping of transgenic mice by PCR analysis. Primers lacZ295U (5′-GAGTGCGATCTTCCTGAGGCCGATACTGTC-3′; SEQ ID NO: 15) and lacZ755L (5′-CGCGGCTGAAATCATCATTAAAGCGAGTGG-3′; SEQ ID NO: 16) were used for detection of the Dmp1 mutant allele (461 bp), and primers DMP-W2 (5′-GCCCCTGGACACTGACCATAGC-3′ SEQ ID NO: 17) and DMP1-W4 (5′-CTGTTCCTCACTCTCACTGTCC-3′ SEQ ID NO: 18) were used for detection of the wild-type allele (˜400 bp). The primers, DMP1326U (5′-CAGCCGTTCTGAGGAAGACAGTG-3′ SEQ ID NO: 19 from Dmp1 cDNA) and SV1738L (5′-TGTCCAAACTCATCAATGTATCT-3′ SEQ ID NO: 20 from SV40 polyA) were used for detection of the Col1a1-Dmp1 transgene or Col1a1-57K transgene and gave rise to a ˜337 by product.

### In Situ Hybridization:

In situ hybridization was done by Anita Xie (School of Dentistry, UMKC). The digoxigenin (DIG)-labeled DMP1 cRNA probe was prepared using a 1.1-kb murine DMP1 cDNA fragment with an RNA Labeling Kit (Roche, Indianapolis, Ind., USA). The 1.1-kb cDNA fragment was obtained by PCR using the full-length DMP cDNA as a template with the following primers, forward primer (5′-CTCCGCAGACACCACACAGTCC-3′; SEQ ID NO: 21) and reverse primer (5′-TAG CCG TCC TGA CAG TCA TTG TC-3′; SEQ ID NO: 22). The PCR product was subsequently cloned into the EcoRI site of the pBluescript SK-vector (Stratagene, La Jolla, Calif., USA). In situ hybridization on paraffin sections was carried out essentially as described previously (Feng et al. 2002). The hybridization temperature was set at 55° C., and washing temperature at 70° C. so that endogenous alkaline phosphatase (AP) would be inactivated. DIG-labeled nucleic acids were detected in an enzyme-linked immunoassay with a specific anti-DIG-AP antibody conjugate and an improved substrate that gives rise to a red signal (Vector Laboratories, Burlingame, Calif., USA) according to the manufacturer's instructions.

### High Resolution Radiography:

The bone samples from Dmp1-null mice, rescued mice as well as control mice were dissected free of muscle. The whole mice or the dissected bones were X-rayed on a Faxitron model MX-20 Specimen Radiography System with a digital camera (Faxitron X-ray Corp., Buffalo Grove, Ill., USA).

### Safranin-O Staining of the Growth Plate Cartilage:

Safranin-O staining was performed to stain proteoglycans in the growth plate of 2-month-old Dmp1-null mice, rescued mice as well as control mice, as described previously (Ye et al. 2005). Briefly, the dissected bones were fixed in 4% paraformaldehyde in 1×PBS and decalcified in Fisher Cal-Ex* decalcifier (Fisher Scientific, Pittsburgh, Pa., USA) overnight. The decalcified bones were embedded in paraffin and sectioned at a thickness of 5 μm. Deparaffinized sections were stained in Weigert's iron hematoxylin, containing 1% hematoxylin, 2% ferric chloride, 0.5% (v/v) concentrated hydrochloric acid and 40% ethanol, for 5 minutes, followed by one dip in 1% acid alcohol. The sections were then incubated sequentially in 0.02% fast green for 1 minute, 1% acetic acid for 30 seconds and 0.1% safranin O for 20 minutes. The stained sections were dehydrated and mounted with permount.

### Immunohistochemistry:

The paraffinized bone sections were deparaffinized and gradually rehydrated, incubated with 0.1% trypsin-0.1% CaCl₂ (pH 7.8) at 37° C. for 30 min for antigen retrieval, and endogenous peroxidase quenched with 5% H₂O₂-PBS. Immunostaining of DMP1 protein was performed as described previously (Ye et al. 2004) using the same DMP1 antibodies used in western blot analysis. Antibody 784 was raised against the N-terminal peptide of DMP1 (116-136), or antibody 785 against the C-terminal peptide (485-499). E11 protein was detected using monoclonal antibody 8.1.1 diluted 1:50, as described previously (Zhang, Barragan-Adjemian, Ye, Kotha, Dallas, Lu, Zhao, Harris, Harris, Feng and Bonewald 2006).

### Double Fluorochrome Labeling for Measurement of Bone Formation Rates:

To examine the bone formation rate in Dmp1 null mice as well as the rescued mice, double fluorescence labeling was performed and analyzed. Briefly, a calcein label (5 mg/kg i.p., Sigma-Aldrich, St. Louis, Mo., USA) was administered to 7-week-old mice. This was followed by injection of an alizarin red label (20 mg/kg i.p., Sigma-Aldrich, St. Louis, Mo., USA) 5 days later. Mice were sacrificed 2 days after injection of the second label, and the ulnae were removed and fixed in 70% ethanol for 2 days until further processing. The specimens were dehydrated through a graded series of ethanol (70-100%) and embedded in methylmethacrylate (MMA, Buehler, Lake Bluff, Ill., USA). One hundred micrometer sections were cut using a Leitz 1600 saw microtome. The unstained sections were viewed under epifluorescent illumination using a Nikon E800 microscope (Nikon, Tokyo, Japan).

### Resin-Casted Scanning Electron Microscopy (SEM):

Resin-casted SEM was performed as described previously (Feng, Ward, Liu, Lu, Xie, Yuan, Yu, Rauch, Davis, Zhang, Rios, Drezner, Quarles, Bonewald and White 2006). Ulnae were dissected and fixed in 4% paraformaldehyde at 4° C. overnight. The bone samples were dehydrated in ascending concentrations of ethanol (from 70% to 100%), embedded in MMA (Buehler, Lake Bluff, Ill., USA), and the surface polished using 1 μm and 0.3 μm alumina alpha micropolish II solution (Buehler) in a soft cloth rotating wheel. The polished surface was acid etched with 37% phosphoric acid for 2-10 seconds, followed by treatment with 5% sodium hypochlorite for 5 minutes. The samples were then coated with gold and palladium, and examined using an FEI/Philips XL30 Field emission environmental scanning electron microscope (Hillsboro, Oreg., USA).

### Visualization of the Lacuno-Canalicular System by Procion Red:

Procion red dye injection was used to give a visual representation of the organization of the lacunar-canalicular system as described previously (Feng et al. 2006). Briefly, procion red was injected 10 minutes prior to sacrifice through the tail vein (0.8% procion red in sterile saline, 10 μl/g body weight) under anesthesia by avertin (5 mg/kg body weight). After sacrifice, the ulnae were fixed in 70% ethanol followed by dehydration and sectioned at 100-μm thickness using a Leitz 1600 saw microtome. The specimens were then viewed under a Nikon C100 confocal microscope (Nikon, Tokyo, Japan) and photographed using an Optronics cooled CCD camera.

### Statistical Analysis:

Statistical differences between groups were conducted by One-way ANOVA with Bonferroni's post test. All values are expressed as means±standard error of the mean. p<0.05 was considered statistically significant. All computations were performed using GraphPad Prism version 3.0a for Macintosh (GraphPad Software, Inc., San Diego, Calif., USA).

### Results

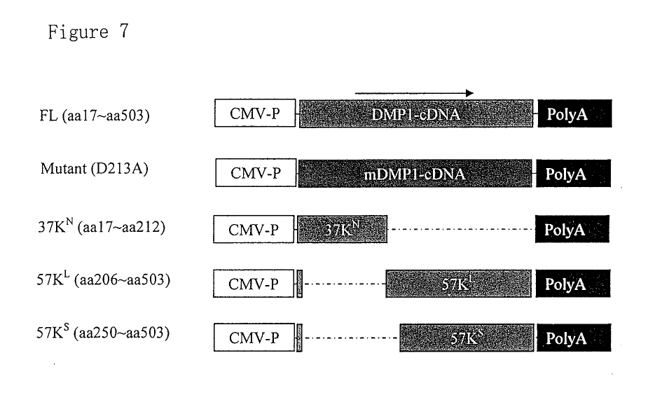

Although the intact native full-length DMP1 has not been isolated from the mineralized tissues, two proteolytic fragments, a 37 kDa N-terminal fragment and a 57 kDa C-terminal fragment, have been isolated from rat long bone and dentin extracts, suggesting that DMP1 might be processed into two fragments in vivo (Qin et al. 2003b). To test the cleavage hypothesis, various forms of DMP1 expression constructs were made (FIG. 7), including a recombinant full-length DMP1, a full-length DMP1 with a cleavage site mutated at amino acid 213 (D-to-A), a 37K N-terminal fragment (aa17 to aa212), and two different forms of the 57 kDa C-terminal fragment (aa206 to aa503, and aa250 to aa503, respectively). Both 57 kDa fragments used the native DMP1 signal peptide, aa1 to aa17 (MKTVILLVFL WGLSCAL).

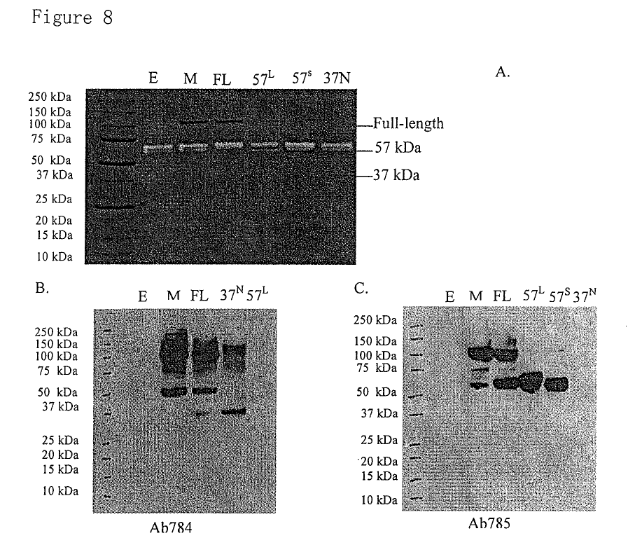

Stains-All stain was used to visualize the various forms of recombinant DMP1 expressed in 293EBNA cells, since DMP1 is a highly acidic phosphorylated extracellular matrix protein (FIG. 8A). Stains-All stains phosphorylated protein blue and stains the non-phosphorylated proteins and background pink. It is of note that the conditioned medium from the 293EBNA cells expressing the pcDNA3 vector shows no blue protein staining. The intact recombinant DMP1 was processed into 37 and 57 kDa fragments equal in size to the recombinant 57 kD C-terminal fragment and 37 kDa N-terminal fragment. In addition, the recombinant DMP1 containing the single cleavage site mutation was not cleaved. The size of the recombinant long form of 57 kDa C-terminal fragment (57K^{L}) was almost identical to the cleaved 57 kDa fragment from the recombinant full-length DMP1. These findings suggest that in 293EBNA cells the recombinant DMP1 was processed at or close to the conserved cleavage site.

The results from Stains-All stain were further confirmed by western-blot analysis using antibody 784 (FIG. 8B), which recognizes the 37 kDa N-terminal fragment as well as the full-length DMP1 and antibody 785 (FIG. 8C), which recognizes the 57 kDa C-terminal fragment as well as full length DMP1. Antibody 784 did not react with either the recombinant nor the cleaved 57 kDa fragment and antibody 785 did not react with either the recombinant nor the cleaved 37 kDa fragment, validating the cleaved fragments as being either the amino- or carboxy-terminal fragments and showing that there was no cross reactivity between two antibodies (FIGS. 8B and C). Recombinant DMP1 was also processed similarly in 2T3 cells, an osteoblastic cell line, and completely cleaved in CHO cells by western-blot analysis (data not shown), suggesting that this cleavage may not be unique to cells that undergo mineralization.

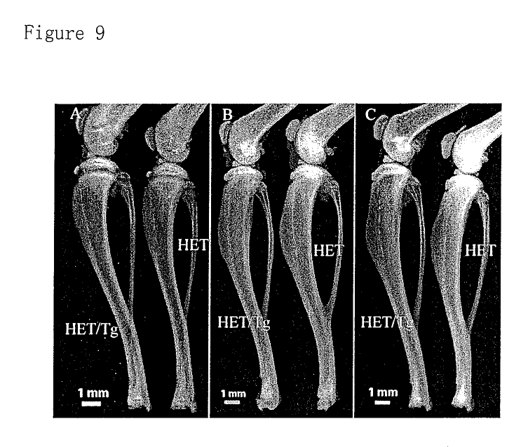

Over-expression of DMP1 using the 3.6-kb Col1a1-DMP1 Transgene Has no Effect on the Skeleton. Dmp1 ablation in mice results in profound skeletal abnormalities postnatally (Ye et al. 2005; Ling et al. 2005), suggesting an essential role of DMP1 in skeletal development. To better understand the in vivo function of DMP1, transgenic mice overexpressing the full-length DMP1 under control of the 3.6-kb Col1a1 promoter were generated, which results in expression of DMP1 in the osteoblast lineage. Four independent mouse lines were obtained for the 3.6-kb Col1a1-DMP1 transgene. Surprisingly, the transgenic mice overexpressing DMP1 on a wild-type background showed no apparent phenotype, as indicated by radiographical or histological examination. Expression of the DMP1 transgene at both the mRNA and protein level was confirmed in the expected locations and showed that expression of the transgene was actually higher than expression of the endogenous Dmp1 gene (Data not shown). Also, overexpression of DMP1 using the Col1a1 promoter in the Dmp1 heterozygote did not result in a skeletal phenotype (FIG. 9). Radiographs of the tibiae showed that the Dmp1 heterozygous mice carrying the Col1a1-DMP1 transgene have no apparent skeletal phenotype compared to the Dmp1 heterozygous mice (HET), at ages of 1 month (FIG. 9A), 2 months (FIG. 9B), and 5 months (FIG. 9C).

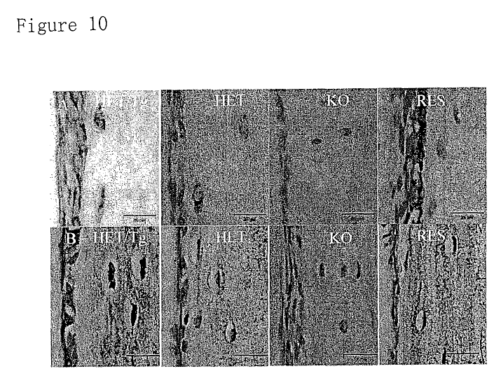

Next, the Dmp1-null mice were used as a genetic background on which to re-express full-length DMP1. FIG. 10 shows that endogenous Dmp1 expression in heterozygous controls was restricted to osteocytes as reported previously (Feng et al. 2003). As expected, Dmp1 expression was undetectable in Dmp1-null mice. In both heterozygous mice or Dmp1-null mice carrying the Col1a1-DMP1 transgene, the expression was actually much higher than control levels and high expression was seen in the osteoblast layer, which does not normally express DMP1.

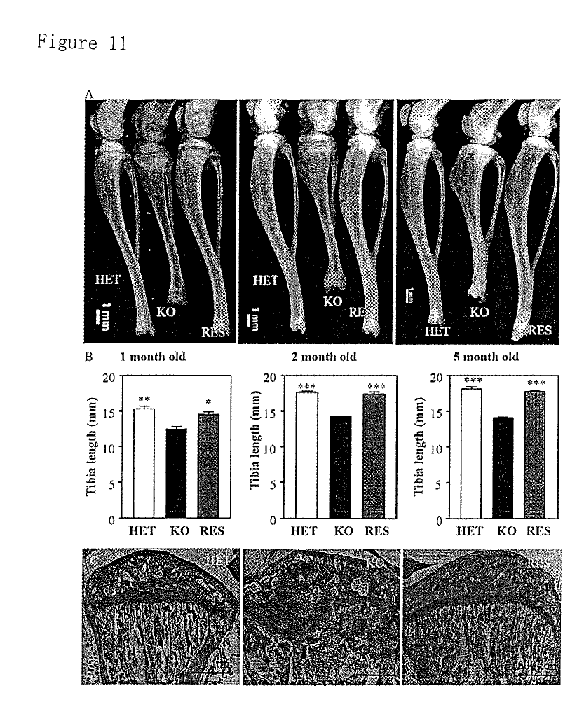

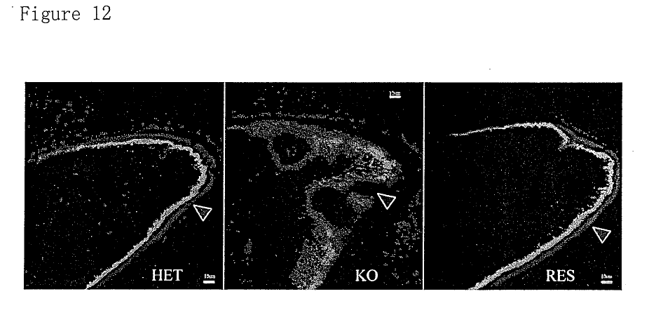

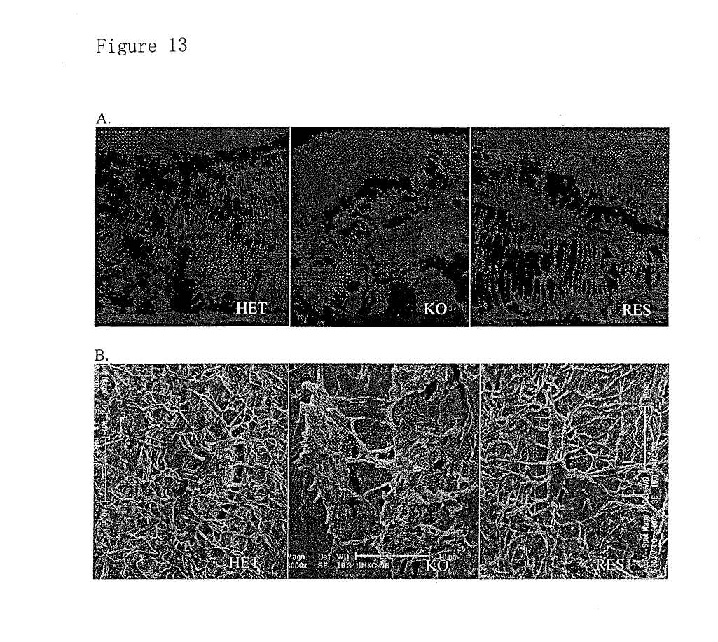

Immunohistochemistry showed that DMP1 protein was present in both osteoblasts and osteocytes in Dmp1 overexpressing or re-expressing mice, suggesting the DMP1 expressed by osteoblasts was carried over to the mature osteocytes.
Re-expression of the Full-length DMP1 Rescues the Skeletal Abnormalities in Dmp1-null mice. Although the mice overexpressing DMP1 on the Dmp1 heterozygous mouse background showed no apparent phenotype with age (FIGS. 9A, B and C), representative radiographs of tibiae from 1-month-old, 2-month-old and 5-month-old mice showed rescue of the rachitic phenotype by targeted expression of DMP1 in Dmp1-null mice (RES), compared to age-matched Dmp1 heterozygous control mice (HET) and Dmp1-null mice (KO) (FIG. 11A). The quantified data show that the length of tibiae was rescued by targeted expression of DMP1 in Dmp1-null mice, and there is no significant difference between the rescued group and the control group at all ages examined (FIG. 11B). Histological examination by Safranin-O staining confirmed that the disorganized growth plate was corrected by the age of 2 months (FIG. 11C). Fluorochrome-labeled sections of the ulnae (FIG. 12) showed sharp, distinct labeling lines in the heterozygous control mice (HET). In the Dmp1-null mice (KO), the fluorochrome labeling appeared more diffuse, suggesting impaired mineral deposition. Expression of the full-length DMP1 in Dmp1-null mice (RES) restored the two discrete labeling lines, confirming that the mineral deposition defects were rescued.
Re-expression of the Full-length DMP1 Restores the Lacuno-canalicunar Morphology in Dmp1-null mice. Procion red is a small molecular dye that will permeate the lacuno-canalicunar system after intravenous administration, giving a visual representation of the lacuno-canalicunar system (FIG. 13A). The lacuno-canalicular system is highly organized, extensively branched and regularly spaced in Dmp1 heterozygous control mice (HET). In contrast, the lacuno-canalicular system in Dmp1-null mice is less branched and appears disorganized. Re-expression of the full-length DMP1 in the Dmp1-null mice (RES) rescued the morphology of the lacuno-canalicular system, similar to the HET controls.

Next, the osteocyte lacuno-canalicular system was examined using scanning electron microscopy of acid-etched resin embedded samples (FIG. 13B) (Martin et al. 1978). With this technique, polished surfaces of the resin embedded bone were acid-etched to remove mineral, leaving a relief cast of the non-mineralized areas that have been infiltrated by resin. This technique therefore shows the architecture of the lacuno-canalicular system. The lacuno-canalicular system appears rough in surface, disorganized, and less branched in Dmp1-null mice (KO), compared to age-matched heterozygous control mice (HET). The abnormal lacuno-canalicular system was rescued by the full-length DMP1 under control of 3.6-kb Col1a1 promoter (RES).

Taken together, these observations suggest that DMP1 plays a critical role in establishing the correct architecture and organization of the lacuno-canalicular system, either as a secondary consequence of its effects on mineralization or through a direct effect of DMP1 on the formation of the walls of the lacunae and canaliculi.

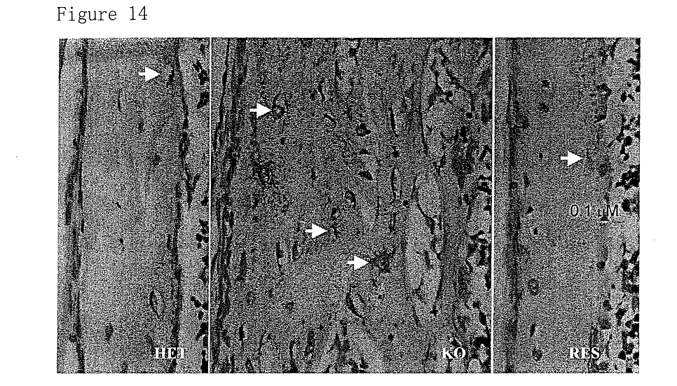

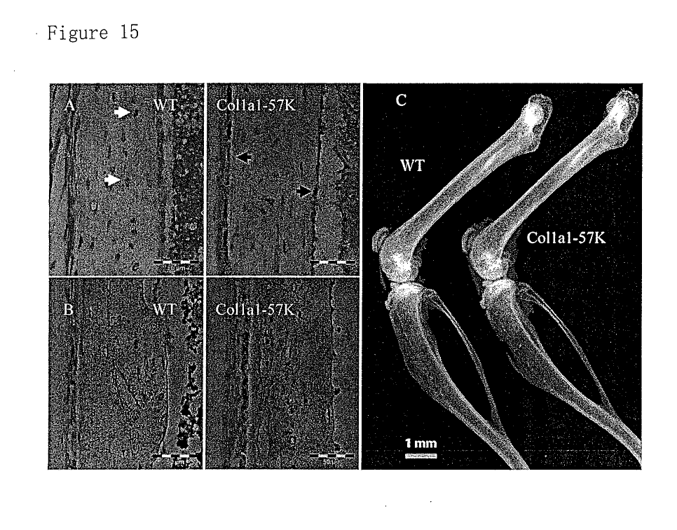

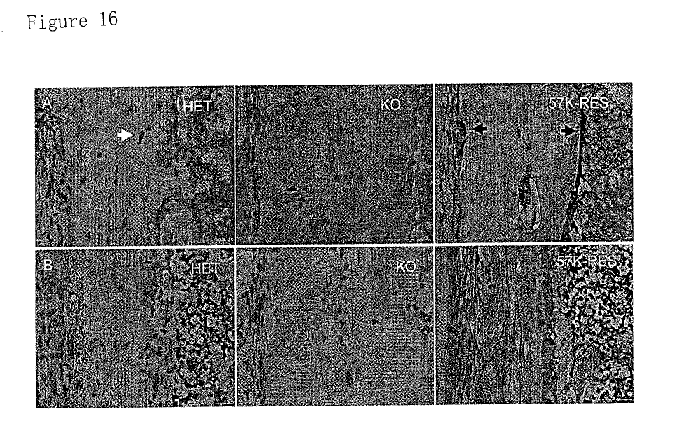

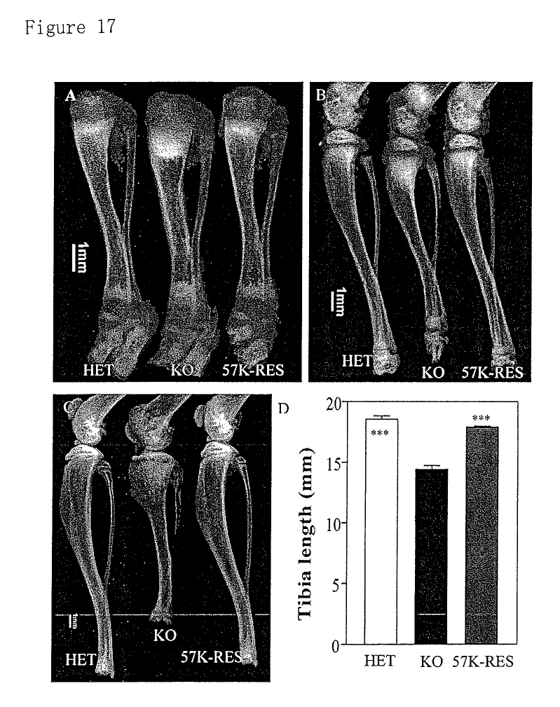

Re-expression of the Full-length DMP1 Rescues Osteoblast Differentiation in the Dmp1-null mice. Next, studies were performed to examine the maturation and differentiation of osteoblasts into osteocytes in the heterozygous control mice (HET), Dmp1-null mice (KO) as well as Dmp1-null mice rescued by Col1a1-DMP1 transgene (RES). Immunohistochemistry (FIG. 14) showed that E11/gp38, an early osteocyte marker, was restricted to the early osteocytes in the control mice, as described previously (Zhang et al. 2006); however, it was elevated in Dmp1-null osteocytes, regardless of whether they were newly formed or deeply embedded, suggesting that the osteocytes in the Dmp1-null mice are trapped in an early differentiated state and do not progress to fully mature osteocytes. Re-expression of the full-length DMP1 in Dmp1-null mice (RES) restored the pattern of E11 expression to the early osteocytes, indicating that the differentiation of osteocytes was restored. These observations suggest that DMP1 expression in the extracellular matrix is essential for differentiation of osteoblasts to fully mature osteocytes.
Overexpression of the 57 kDa C-terminal Fragment Driven by the 3.6-kb Col1a1 Promoter has No Apparent Phenotype. To test the hypothesis that the carboxy-57 kDa fragment is the functional domain of DMP1, the same 3.6-kb Col1a1 promoter was used to generate transgenic mice overexpressing the long form of the 57 kDa C-terminal fragment (57K^{L}) in the osteoblast lineage. Ten founders were generated and partially characterized by in situ hybridization and immunohistochemistry. Five independent transgenic mouse lines had various levels of transgene expression in osteoblast lineage. Three transgenic lines were further evaluated based on their expression levels. The results from one line are presented in this study. Similar to the expression pattern of Col1a1 full-length DMP1 mice, the mRNA of the 57 kDa transgene was expressed in the osteoblast lineage as shown by in situ hybridization (FIG. 15A), and the protein of this transgene was present in both osteoblasts and osteocytes as determined by immunohistochemistry (FIG. 15B). Both mRNA and protein levels of the transgene were again much higher, compared to age-matched wild-type controls. However, none of transgenic mice overexpressing the 57 kDa fragment on the wild-type background showed any apparent skeletal phenotype, compared to the WT mice (FIG. 15C).
Expression of the 57 kDa C-terminal Fragment Rescues Skeletal Abnormalities in the Dmp1-null Mice. Next, the Col1a1-57K transgene was expressed in the Dmp1-null mice by crossing the Col1a1-57K transgenic mice with Dmp1-null mice. In situ hybridization and immunohistochemistry (FIG. 16) showed that expression of endogenous DMP1 in Dmp1 heterozygous mice (HET) was found predominantly in the osteocytes embedded in the bone matrix, as reported previously (Feng et al. 2003). No expression of endogenous DMP1 was detected in Dmp1-null mice (KO). In situ hybridization (FIG. 16A) showed that the Col1a1-57K transgene was highly expressed in osteoblasts as expected, however, the immunohistochemistry (FIG. 16B) indicated that the 57 kDa fragment was present in the matrix surrounding osteoblasts and osteocytes, identical to the expression pattern of the full-length DMP1 transgene. Importantly, the representative radiographs of the tibiae from 10-day-old mice (FIG. 17A), 3-week-old mice (FIG. 17B) and 7-week-old mice (FIG. 17C) showed that expression of the 57 kDa fragment rescues the rachitic bone phenotype of Dmp1-null mice (RES), compared to age-matched Dmp1-null mice (KO), and Dmp1 heterozygous control mice (HET). The quantified data confirmed that the length of tibiae was rescued by targeted expression of the 57 kDa C-terminal fragment, and there was no significant difference between the 57 kDa fragment rescued group and the control group at the age of 7 weeks (FIG. 17D). These data strongly support that the 57 kDa C-terminal fragment is the essential functional domain of DMP1.

1. A method for screening patients to identify individuals suffering from autosomal recessive hypophosphatemic rickets (ARHR) or who are at risk of producing offspring that suffer from autosomal recessive hypophosphatemic rickets (ARHR), said method comprising the steps of
analyzing the DMP1 sequences of the patient to determine if the patient has a defective DMP1 gene, wherein the detection of a defective DMP1 gene is associated with autosomal recessive hypophosphatemic rickets.

2. The method of claim 1 wherein the DMP1 sequences are analyzed by sequencing at least one region of the DMP1 coding region.

3. The method of claim 2 wherein the region sequenced comprises the nucleic acid sequences encoding the 57 kDa fragment of DMP1.

4. The method of claim 2 wherein the region to be sequenced is selected from the regions comprising the nucleic acid sequences of SEQ ID NO: 38 and SEQ ID No: 44.

5. The method of claim 1 wherein the DMP1 sequences are analyzed by PCR amplification and melting curve analysis.

6. The method of claim 5 wherein the amplified region corresponds to the region comprising SEQ ID NO: 38 and SEQ ID No: 44 in the wild type sequence.

7. The method of claim 4 wherein the specified region to be sequenced is amplified by PCR prior to sequencing the region.

8. The method of claim 1 wherein the entire DMP1 coding sequence is sequenced.

9. The method of claim 1 wherein the DMP1 sequences are analyzed by hybridization with nucleic acid probes that are specific for defective DMP1 genes.

10. The method of claim 9 wherein the probe comprises a nucleic acid sequence selected from the group consisting of SEQ ID NO: 39 and SEQ ID NO: 40.

11. The method of claim 10 wherein the probe is labeled.

12. The method of claim 11 wherein the probe serves as one member of a pair of PCR primers.

13. A kit for screening biological samples for the presence of defective DMP1 genes, said kit comprising a first pair of PCR primers for amplifying at least one region of the DMP1 gene.

14. The kit of claim 13 further comprising an additional pair of PCR primers for amplifying a different regions of the DMP1 gene than the first pair of PCR primers.

15. A monoclonal antibody that specifically binds to the variant DMP1 protein of SEQ ID NO: 42 or SEQ ID NO: 43.

16. The monoclonal antibody of claim 15 wherein the antibody specifically binds to the protein of SEQ ID NO: 42.


SEQUENCE LISTING (46 sequences)

SEQ ID NO: 1 - DNA, 20 bp, Artificial Sequence
  PCT primer
ctgctagagcctatccggac

SEQ ID NO: 2 - DNA, 19 bp, Artificial Sequence
  PCT primer
agtgatgcttctgcgacaa

SEQ ID NO: 3 - DNA, 20 bp, Artificial Sequence
  PCT primer
ggtgtgaaccacgagaaata

SEQ ID NO: 4 - DNA, 20 bp, Artificial Sequence
  PCT primer
tgaagtcgcaggagacaacc

SEQ ID NO: 5 - DNA, 22 bp, Artificial Sequence
  PCT primer
gaatcgcatcccaatatgaaga

SEQ ID NO: 6 - DNA, 22 bp, Artificial Sequence
  PCT primer
cccaagactccgtcctgtgaga

SEQ ID NO: 7 - DNA, 42 bp, Artificial Sequence
  PCT primer
ttgggggctgtcctgtgctctcgatgatgaagggatgcagag

SEQ ID NO: 8 - DNA, 40 bp, Artificial Sequence
  PCT primer
ttgggggctgtcctgtgctctcgatgacagccagtctgtg

SEQ ID NO: 9 - DNA, 22 bp, Artificial Sequence
  PCT primer
catcggtcgacggatccttatc

SEQ ID NO: 10 - DNA, 63 bp, Artificial Sequence
  PCT primer
gaatcgatatcccaatatgaagactgtcattctccttgtgttcctttgggggctgtcctgtgc

SEQ ID NO: 11 - DNA, 30 bp, Artificial Sequence
  PCT primer
gtgatatcgaatcgcatcccaatatgaaga

SEQ ID NO: 12 - DNA, 32 bp, Artificial Sequence
  PCT primer
tctctagagctagctctgcatcccttcatcat

SEQ ID NO: 13 - protein, 21 aa, Mus musculus
GLGPEEGQWGGPSKLDSDEDS

SEQ ID NO: 14 - protein, 15 aa, Mus musculus
AYHNKPIGDQDDNDC

SEQ ID NO: 15 - DNA, 30 bp, Artificial Sequence
  PCT primer
gagtgcgatcttcctgaggccgatactgtc

SEQ ID NO: 16 - DNA, 30 bp, Artificial Sequence
  PCT primer
cgcggctgaaatcatcattaaagcgagtgg

SEQ ID NO: 17 - DNA, 22 bp, Artificial Sequence
  PCT primer
gcccctggacactgaccatagc

SEQ ID NO: 18 - DNA, 22 bp, Artificial Sequence
  PCT primer
ctgttcctcactctcactgtcc

SEQ ID NO: 19 - DNA, 23 bp, Artificial Sequence
  PCT primer
cagccgttctgaggaagacagtg

SEQ ID NO: 20 - DNA, 23 bp, Artificial Sequence
  PCT primer
tgtccaaactcatcaatgtatct

SEQ ID NO: 21 - DNA, 22 bp, Artificial Sequence
  PCT primer
ctccgcagacaccacacagtcc

SEQ ID NO: 22 - DNA, 23 bp, Artificial Sequence
  PCT primer
tagccgtcctgacagtcattgtc

SEQ ID NO: 23 - protein, 25 aa, Reptile
RKLMLDVYHNKPIGDYDDNDCQDGY

SEQ ID NO: 24 - protein, 25 aa, Mus musculus
RKLALDAYHNKPIGDQDDNDCQDGY

SEQ ID NO: 25 - protein, 25 aa, Bos taurus
RKLVLDAYHNKPIGDQDDNDCQDGY

SEQ ID NO: 26 - protein, 25 aa, Sus scrofa
RKLILDAYHNKPIGDQDDNDCQDGY

SEQ ID NO: 27 - protein, 25 aa, Homo sapiens
RKLILDAYHNRPMGDQDDNDCQDGY

SEQ ID NO: 28 - protein, 40 aa, Mus musculus
RKLILDATNPLGTKMTMTAKTAISISCPKKQLSHKGVLGT

SEQ ID NO: 29 - protein, 32 aa, Reptile
MKTVLVLISLWALTYAHPVPNHPNVHDGSSKE

SEQ ID NO: 30 - protein, 32 aa, Mus musculus
MKTVIVLVFLWGLSCALPVARYHNTESESSEE

SEQ ID NO: 31 - protein, 32 aa, Bos taurus
MKTTILLMFLWGLSCALPVARYQNTKSKSSEE

SEQ ID NO: 32 - protein, 32 aa, Sus scrofa
MKTSILLMLLWGLSCALPVARYQNTKSKSSEE

SEQ ID NO: 33 - protein, 32 aa, Homo sapiens
MKISILLMFLWGLSCALPVTRYQNNESEDSEE

SEQ ID NO: 34 - protein, 25 aa, Mus musculus
MFLWGLSCALPVTRYQNNESEDSEE

SEQ ID NO: 35 - DNA, 7 bp, Mus musculus
ctatcac

SEQ ID NO: 36 - DNA, 15 bp, Homo sapiens
ccaactgtgaagatc

SEQ ID NO: 37 - DNA, 16 bp, Homo sapiens
gttgatgcaacaaacc

SEQ ID NO: 38 - DNA, 23 bp, Homo sapiens
gttgatgcctatcacaacaaacc

SEQ ID NO: 39 - DNA, 6 bp, Homo sapiens
tgcaac

SEQ ID NO: 40 - DNA, 8 bp, Homo sapiens
atgcaaca

SEQ ID NO: 41 - protein, 513 aa, Homo sapiens
MKISILLMFLWGLSCALPVTRYQNNESEDSEEWKGHLAQAPTPPLESSESSEGSKVSSEEQANEDPSDSTQSEEGLGSDDHQYIYRLAGGFSRSTGKGGDDKDDDEDDSGDDTFGDDDSGPGPKDRQEGGNSRLGSDEDSDDTIQASEESAPQGQDSAQDTTSESRELDNEDRVDSKPEGGDSTQESESEEHWVGGGSDGESSHGDGSELDDEGMQSDDPESIRSERGNSRMNSAGMKSKESGENSEQANTQDSGGSQLLEHPSRKIFRKSRISEEDDRSELDDNNTMEEVKSDSTENSNSRDTGLSQPRRDSKGDSQEDSKENLSQEESQNVDGPSSESSQEANLSSQENSSESQEEVVSESRGDNPDPTTSYVEDQEDSDSSEEDSSHTLSHSKSESREEQADSESSESLNFSEESPESPEDENSSSQEGLQSHSSSAESQSEESHSEEDDSDSQDSSRSKEDSNSTESKSSSEEDGQLKNIEIESRKLTVDAYHNKPIGDQDDNDCQDGY

SEQ ID NO: 42 - protein, 528 aa, Homo sapiens
MKISILLMFLWGLSCALPVTRYQNNESEDSEEWKGHLAQAPTPPLESSESSEGSKVSSEEQANEDPSDSTQSEEGLGSDDHQYIYRLAGGFSRSTGKGGDDKDDDEDDSGDDTFGDDDSGPGPKDRQEGGNSRLGSDEDSDDTIQASEESAPQGQDSAQDTTSESRELDNEDRVDSKPEGGDSTQESESEEHWVGGGSDGESSHGDGSELDDEGMQSDDPESIRSERGNSRMNSAGMKSKESGENSEQANTQDSGGSQLLEHPSRKIFRKSRISEEDDRSELDDNNTMEEVKSDSTENSNSRDTGLSQPRRDSKGDSQEDSKENLSQEESQNVDGPSSESSQEANLSSQENSSESQEEVVSESRGDNPDPTTSYVEDQEDSDSSEEDSSHTLSHSKSESREEQADSESSESLNFSEESPESPEDENSSSQEGLQSHSSSAESQSEESHSEEDDSDSQDSSRSKEDSNSTESKSSSEEDGQLKNIEIESRKLTVDATNPLGTKMTMTAKTAISISCPKKQLSHKGVLGT

SEQ ID NO: 43 - protein, 513 aa, Homo sapiens
VKISILLMFLWGLSCALPVTRYQNNESEDSEEWKGHLAQAPTPPLESSESSEGSKVSSEEQANEDPSDSTQSEEGLGSDDHQYIYRLAGGFSRSTGKGGDDKDDDEDDSGDDTFGDDDSGPGPKDRQEGGNSRLGSDEDSDDTIQASEESAPQGQDSAQDTTSESRELDNEDRVDSKPEGGDSTQESESEEHWVGGGSDGESSHGDGSELDDEGMQSDDPESIRSERGNSRMNSAGMKSKESGENSEQANTQDSGGSQLLEHPSRKIFRKSRISEEDDRSELDDNNTMEEVKSDSTENSNSRDTGLSQPRRDSKGDSQEDSKENLSQEESQNVDGPSSESSQEANLSSQENSSESQEEVVSESRGDNPDPTTSYVEDQEDSDSSEEDSSHTLSHSKSESREEQADSESSESLNFSEESPESPEDENSSSQEGLQSHSSSAESQSEESHSEEDDSDSQDSSRSKEDSNSTESKSSSEEDGQLKNIEIESRKLTVDAYHNKPIGDQDDNDCQDGY

SEQ ID NO: 44 - DNA, 15 bp, Homo sapiens
ccaactatgaagatc

SEQ ID NO: 45 - protein, 33 aa, Homo sapiens
TNPLGTKMTMTAKTAISISCPKKQLSHKGVLGT

SEQ ID NO: 46 - protein, 17 aa, Mus musculus
MKTVILLVFLWGLSCAL